(19) United States
(12) Patent Application Publication
(10) Pub. No.: US 2011/0243958 A1
(43) Pub. Date: Oct. 6, 2011

(54) ANTIMICROBIAL AGENTS THAT TARGET BACTERIAL VKOR

(75) Inventors: Jonathan Beckwith, Cambridge, MA (US); Rachel Dutton, Cambridge, MA (US); Dana Boyd, Medford, MA (US); Mehmet Berkmen, Somerville, MA (US)

(73) Assignee: PRESIDENT AND FELLOWS OF HARVARD COLLEGE, Cambridge, MA (US)

(21) Appl. No.: 13/123,470

(22) PCT Filed: Oct. 14, 2009
(86) PCT No.: PCT/US09/60708

§ 371 Date: Jun. 21, 2011

Related U.S. Application Data
(60) Provisional application No. 61/105,668, filed on Oct. 15, 2008.

(51) Int. Cl. A01N 37/18; A01N 43/90; A01N 43/16; C12Q 1/26; C12Q 1/42; C12Q 1/34; A01P 1/00

(52) U.S. Cl. 424/158.1; 514/2.3; 514/44 A; 514/457; 435/25; 435/21; 435/18

(57) ABSTRACT

Aspects of the invention relate to a method for inhibiting the growth of a microbe that expresses bacterial vitamin K epoxide reductase (bVKOR). The method involves contacting the bacterial cell with an effective amount of an agent that inhibits bVKOR. Agents include a drug, ligand or portion thereof, protein, polypeptide, small organic molecule, antisense nucleic acid, RNAi, or antibody. Examples of useful agents are a phenylpropanoid, a modified phenylpropanoid, a coumarin or modified coumarin. A particularly useful agents is warfarin or a variant thereof or ferulenol or a variant thereof. The microbe is any microbe carrying a bVKOR gene, such as Mycobacterium tuberculosis.

### CROSS REFERENCE TO RELATED APPLICATIONS

The present application claims priority benefit under 35 U.S.C. §119(e) of provisional application U.S. Ser. No. 61/105,668 filed Oct. 15, 2008.

### GOVERNMENT SUPPORT

This invention was supported by the National Institutes of General Medical Sciences Grant No. #GM41883, and the Government of the United States has certain rights thereto.

### FIELD OF THE INVENTION

The present invention relates to the identification of new classes of antimicrobial agents and anticoagulation agents for therapeutics.

### BACKGROUND

Folding and stability of many proteins requires formation of disulfide bonds-chemical bonds between two cysteines. Although disulfide bond formation was thought to be a spontaneous process, in 1991 it was discovered that the bacterium Escherichia coli has an enzyme, DsbA, required for this process. Subsequent studies showed that eukaryotes also require an enzyme for disulfide bond formation; the ER enzyme PDI catalyzes this process. DsbA and PDI are members of the thioredoxin protein family, sharing a similar structure and a Cys-X-X-Cys active site motif. When DsbA is oxidized, with its two cysteines joined in a disulfide bond, it can donate the disulfide bond to many protein substrates containing cysteines. DsbA is itself reoxidized by a cytoplasmic membrane protein, DsbB. Electrons passed from DsbA to DsbB are then transferred to membrane-localized quinones and ultimately to oxygen. Eukaryotes also have an ER protein, Ero1, to regenerate active PDI.

The spread of multiple drug resistant microbial pathogens (e.g., M. tuberculosis) is an enormous public health problem. The development of antimicrobial agents that have unique targets within the pathogens is needed to facilitate treatment of the multiple drug resistant diseases.

### SUMMARY OF THE INVENTION

Aspects of the invention relate to a method for inhibiting the growth of a microbe that expresses bacterial vitamin K epoxide reductase (bVKOR). The method comprises contacting the bacterial cell with an effective amount of an agent that inhibits bVKOR. In one embodiment, the agent does not detrimentally inhibit human (h)VKOR. In one embodiment, the agent is a drug, ligand or portion thereof, protein, polypeptide, small organic molecule, antisense nucleic acid, RNAi, or antibody. In one embodiment, the agent is a phenylpropanoid, a modified phenylpropanoid, a coumarin or modified coumarin. In one embodiment, the agent is warfarin or a variant thereof or ferulenol or a variant thereof. In one embodiment, the microbe is a microbe identified herein as carrying a bVKOR gene. In one embodiment, the microbe is Mycobacterium tuberculosis. In one embodiment, the agent is identified by the methods disclosed herein.

Aspects of the invention relate to a method for identifying a bVKOR inhibitory agent. The method comprises the steps, testing one or more test agents in a disulfide bond formation assay, wherein bVKOR functions as the oxidant of DsbA in the assay, and identifying test agents that significantly inhibit disulfide bond formation in the assay, wherein the ability of the candidate agent to significantly inhibit disulfide bond formation in the assay indicates that it is a bVKOR inhibitory agent. In one embodiment, the bVKOR is from a microbe identified herein as carrying a bVKOR gene. In one embodiment, the bVKOR is from M. tuberculosis. In one embodiment, the method further comprises testing the test agent identified in the second step in an assay for disulfide bond formation, wherein hVKOR functions as the oxidant of DsbA in the assay, to thereby identify a bVKOR inhibitory agent that does not significantly inhibit hVKOR. In one embodiment, the method comprises testing the test agent identified in the second step in a second assay for bVKOR activity to further indicate the test agent is a bVKOR inhibitory agent. In one embodiment, the second assay for bVKOR activity is a growth inhibitory assay for a microbe that naturally expresses bVKOR.

Aspects of the present invention relate to a method for identifying an antimicrobial agent. The method comprises the steps assaying test agents for bVKOR inhibitory activity, and for hVKOR inhibitory activity, to thereby identify a test agent that inhibits bVKOR significantly more than it inhibits hVKOR, and further assaying the test agent identified, for growth inhibition activity on a bVKOR expressing microbe, wherein an agent that exhibits growth inhibition activity on the microbe, is thereby identified as an antimicrobial agent. In one embodiment, bVKOR inhibitory activity is assayed in a disulfide bond formation assay, wherein bVKOR functions as the oxidant of DsbA in the assay, and wherein hVKOR inhibitory activity is assayed in a disulfide bond formation assay, wherein hVKOR functions as the oxidant of DsbA in the assay. In one embodiment, the method further comprises assaying the identified test agent of the first step for anti-coagulant activity, wherein a test agent which lacks anti-coagulant activity is further assayed in the second step.

Aspects of the present invention relate to a method for identifying a candidate anticoagulation agent. The method comprises the steps testing one or more test agents in a disulfide bond formation assay, wherein hVKOR functions as the oxidant of DsbA in the assay, and identifying test agents that significantly inhibit disulfide bond formation in the assay, wherein the ability of the test agent to significantly inhibit disulfide bond formation in the assay indicates that it is a candidate anticoagulation agent. In one embodiment, the method further comprises testing the identified candidate anticoagulation agents in an anti-coagulation assay, to thereby identify anticoagulation agents. In one embodiment, the hVKOR is wild type hVKOR, or is a polymorphism of hVKOR associated with warfarin resistance.

In one embodiment of the disclosed methods, the test agent is a drug, ligand or portion thereof, protein, polypeptide, small organic molecule, antisense nucleic acid, RNAi, or antibody. In one embodiment of the disclosed methods, the test agent is a phenylpropanoid, a modified phenylpropanoid, a coumarin or modified coumarin. In one embodiment of the disclosed methods, the test agent is warfarin or a variant thereof or ferulenol or a variant thereof. In one embodiment of the disclosed methods, the disulfide bond formation assay is a motility assay. In one embodiment of the disclosed methods the disulfide bond formation assay is a β-gal assay using β-gal fused to a bacterial membrane protein. In one embodiment of the disclosed methods, the β-gal is fused to bacterial membrane protein MalF, to thereby produce a MalF-β-gal fusion protein. In one embodiment of the disclosed methods, the disulfide bond formation assay is an alkaline phosphatase assay. In one embodiment of the disclosed methods, the disulfide bond formation assay is performed in E. coli.

Aspects of the invention relate to an antimicrobial agent identified by one or more of the methods disclosed herein. Aspects of the invention relate to an anticoagulation agent identified by one or more of the method disclosed herein.

### BRIEF DESCRIPTION OF THE DRAWINGS

FIG. 1 is a schematic of the disulfide bond formation pathway of E. coli. (arrows indicate flow of electrons)

FIGS. 2A and B are a pair of graphs representing data the indicates that exported proteins show a unique bias for even numbers of cysteines. FIG. 2A is a line graph that shows cysteine distribution in E. coli K12 proteins—cytoplasmic and exported (classes 1 and 5). FIG. 2B is a graphical representation of counting of all amino acids in E. coli K12 exported proteins. The z-score for the fraction of exported proteins with even numbers of an amino acid (Efrac), is plotted against the AApref for each amino acid (an AApref<1.0 indicates a bias against incorporation of the amino acid into exported proteins). The graph is divided into two regions, A and B. The data in region A indicates that there are significantly more even numbers of the amino acid in exported proteins than is predicted by the random model. The data in region B indicates that exported proteins do not have a significant bias for even numbers of these amino acids (2.57>z>−2.57).

FIG. 3 is a schematic representation of the combined results of disulfide predictions based on cysteine counting and homology searches. Genomes with significant numbers of exported proteins with even numbers of cysteines (z-score>2.57) are indicated by the shading of the inner most ring lining the circle, and the distribution of DsbA (indicated by the next external ring lining the circle) and DsbB (indicated by the third external ring lining the circle) homologs are shown in a representative subset of all organisms analyzed. The genomes containing a homolog of VKOR are indicated by shading at the most external ring lining the circle.

FIG. 4 is a photograph of experimental results of disulfide bond formation assays using motility plates. The data indicates that a bacterial VKOR homolog restores disulfide bond formation to E. coli deleted for dsbB. Disulfide bond formation was assayed using motility plates, as motility requires active disulfide bond formation. Expression of the VKOR homolog from M. tuberculosis restores motility to an E. coli ΔdsbB strain, but not a ΔdsbAΔdsbB strain.

FIG. 5 is a photographs of experimental results from an alkylation assay. The results indicate that the E. coli protein LivK does not become disulfide bonded when expressed in B. fragilis. Determination of redox state of the E. coli protein LivK-myc expressed in B. fragilis, using alkylation. Samples were TCA precipitated then treated as follows, Lane 1: DTT was added to fully reduce the sample to provide a control for unlabelled protein, Lane 2: Mal-PEG alkylation. If the cysteines in the protein are not disulfide bonded, they will react with the 2 kD alkylating agent, resulting in an increase in molecular weight. The arrow indicates the position of the expected shift as a result of alkylation of both cysteines, Lane 3: Control for full alkylation of the protein, samples were first reduced with DTT, then alkylated with Mal-PEG. The asterisk indicates a cross-reacting band, which also shifts upon alkylation (not shown).

FIGS. 6A and B show the bacterial VKOR sequences. FIG. 6A shows the amino acid sequences of M. tuberculosis VKOR homolog (SEQ ID NO: 1). FIG. 6B shows the nucleic acid sequences of M. tuberculosis VKOR homolog (SEQ ID NO: 2).

FIG. 7 is a table of Cysteine distribution in E. coli K12 cysteine-containing proteins. Only exported proteins (class 5) and periplasmic portions of transmembrane proteins (class 4) show a significant bias for even numbers of cysteines.

FIG. 8 is a table listing microbes identified as carrying a bacterial VKOR homolog gene.

### DETAILED DESCRIPTION

Aspects of the present invention relate to the isolation of the microbial vitamin K epoxide reductase (VKOR) homolog genes and expression of the encoded VKOR protein, and to the determination that expression of this gene is necessary for disulfide bond formation in various bacteria (e.g. Mycobacterium tuberculosis). The isolated bacterial VKOR (bVKOR) gene has also been cloned and expressed in other organisms which do not naturally contain a bVKOR gene (e.g. E. coli), and has been found to complement their endogenous disulfide bond formation cellular machinery. This system has allowed for the development of highly sensitive assay systems for the function of bVKOR in an organism in which it is not required for growth. This assay system allows for the rapid screening of test agents to identify agents that inhibit bVKOR. Further, evidence which indicates high conservation of bVKOR function with respect to the human VKOR (hVKOR) sequences, indicates that hVKOR will also complement the endogenous disulfide bond formation cellular machinery of non-bVKOR expressing organisms such as E. coli. This allows the use of hVKOR in an assay for rapid screening of test agents as well, to identify agents which inhibit bVKOR that do not detrimentally inhibit hVKOR. Furthermore, hVKOR can be similarly used in a screening assay to identify agents which inhibit hVKOR, and thereby function as anti-coagulation agents in vertebrates, thereby allowing the identification of new categories/species of therapeutic anticoagulants.

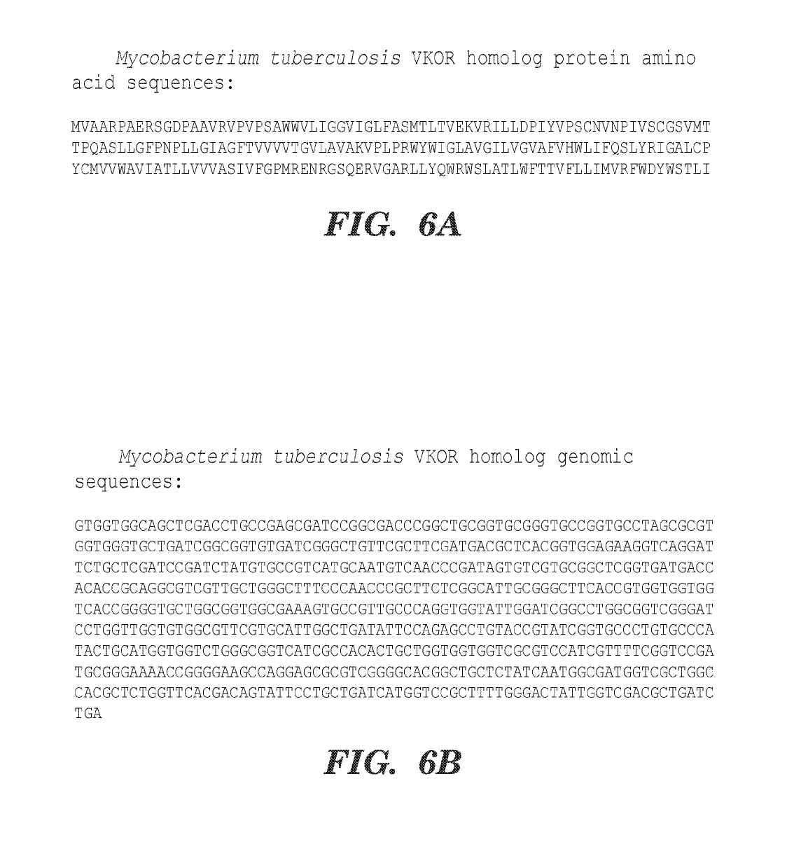

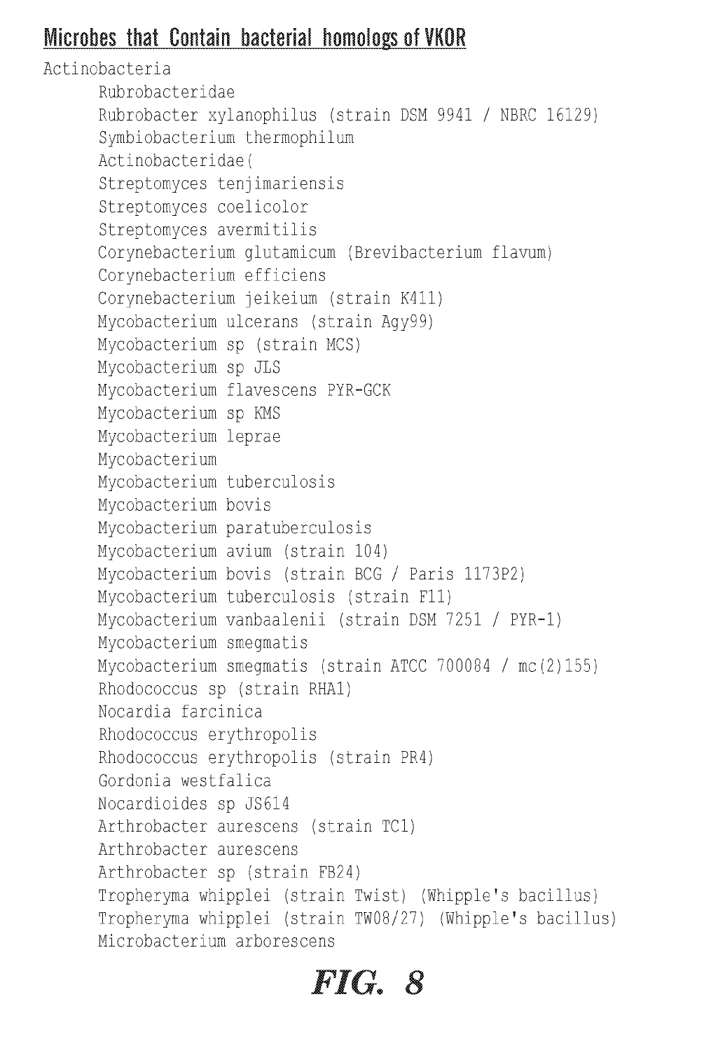

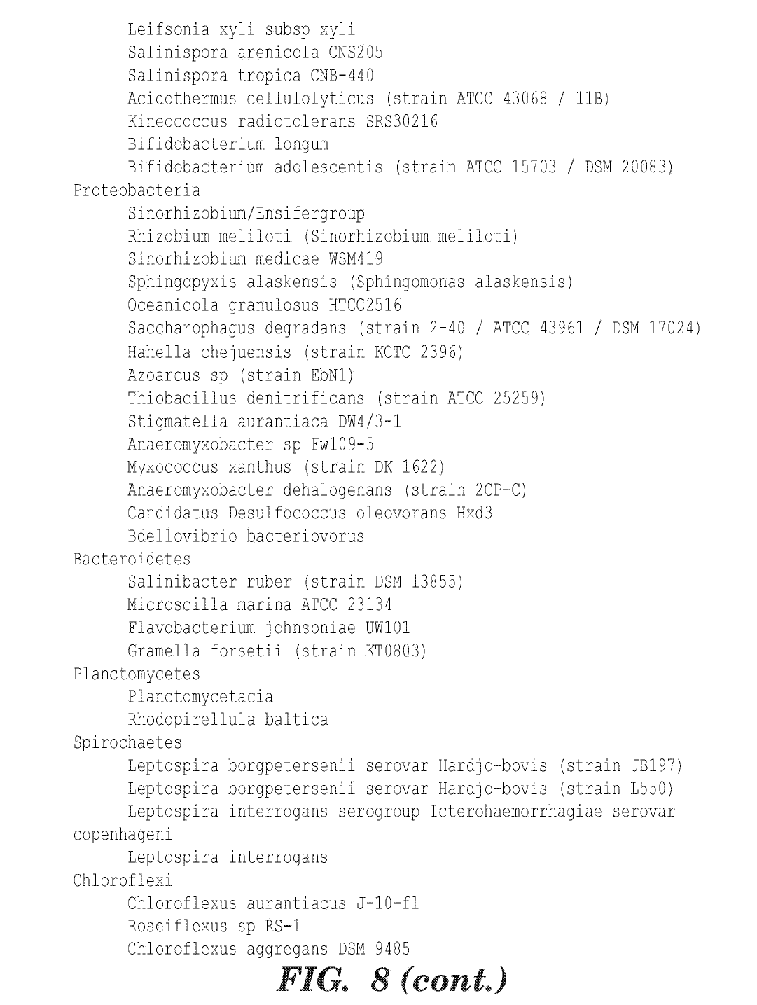

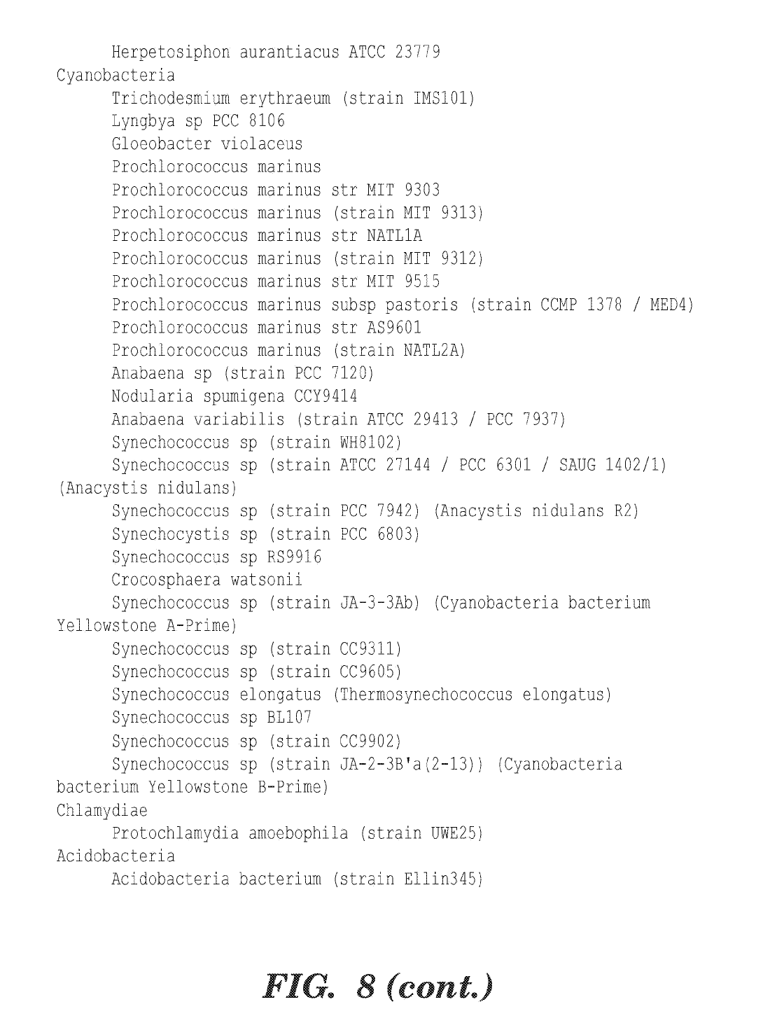

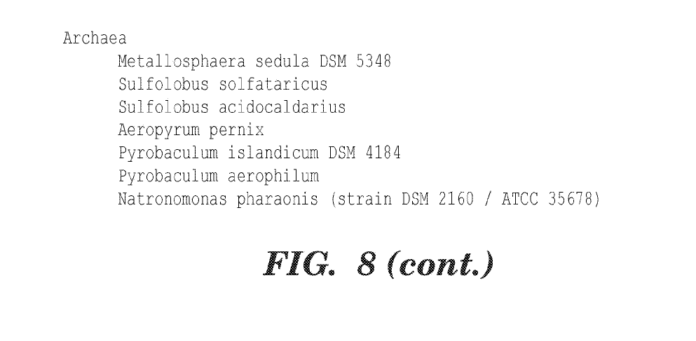

As the term is used herein, “bacterial VKOR” or “bVKOR” refers to the bacterial homolog of human VKOR that is identified as contained in a variety of microbes (Dutton et al., PNAS 105: 11933-11938 (2008)), such as the microbes identified in FIG. 8 herein. One example of bacterial VKOR is Mycobacterial tuberculosis VKOR. The amino acid sequences of M. tuberculosis VKOR is shown in FIG. 6A, and the nucleic acid sequences of the M. tuberculosis gene encoding the VKOR homolog is shown in FIG. 6B.

One aspect of the present invention relates to a method for inhibiting the growth of a microbe (e.g., bacteria) that expresses bVKOR. The method comprises contacting the microbial cell with an effective amount of an agent that inhibits bVKOR. The identification of such an agent is described herein. An effective amount of the agent is an amount sufficient to cause a statistically significant inhibition of growth of the microbe. In one embodiment, the amount is sufficient to completely inhibit all detectable growth. Significant benefit is expected to be produced even under conditions where growth of the microbe is less than completely inhibited. In one embodiment, the amount is sufficient to reduce growth by at least 50% the growth rate. In another embodiment, the amount is sufficient to reduce the growth by at least 60, 70, 80, 90, or 95% of the growth rate. Determination of microbial growth can be performed by the skilled artisan by methods known in the art.

In one embodiment, the agent does not detrimentally inhibit human VKOR (hVKOR). Detrimental inhibition of hVKOR is inhibition of hVKOR sufficiently to cause life-threatening anti-coagulation in a mammalian subject (e.g., a human) to whom the agent is administered in an effective amount to inhibit microbial growth. A therapeutically effective amount, as the term is used herein, is an amount sufficient to inhibit microbial growth, without detrimental inhibition of hVKOR.

Agents with the desired activity are identified by the methods described herein. An agent can be any kind of molecule or complex (e.g., a drug, ligand or portion thereof, protein or polypeptide, small organic molecule, antisense nucleic acid, RNAi, or an antibody).

In one embodiment, the agent has such antimicrobial activity that it completely inhibits microbial growth of a pathogen when administered to a subject in a therapeutically effective amount. Agents which have antimicrobial activity that does not completely inhibit growth when administered in a therapeutically effective amount, are also considered to be of significant value. This is in part, due to the ability of such an agent to augment the activity or effectiveness of a second antibiotic when used in combination (e.g., administered therapeutically). In one embodiment, the agent is used in combination (e.g., administered together or separately into the same subject) with other agents (e.g., known or suspected antibiotics or antimicrobial agents) to significantly reduce the microbial growth of a drug resistant pathogen. In one embodiment, the combined administration completely inhibits microbial growth of an infecting pathogen. Other such antibiotics for use with the agents identified herein are known in the art.

As used herein, an “effective amount” of the agent to be contacted is an amount which delivers sufficient agent to the microbe to produce a detectable amount of growth inhibition. In one embodiment, the amount used produces complete growth inhibition. However, amounts that produce less than complete growth inhibition are also encompassed. Detection of growth inhibition can be by any growth assay known.

The contacting of the agent to the microbe can occur in vivo or in vitro. Contacting in vitro can be, for example, in culture of the microbe, or can be in a culture of cells or organism in which the microbe is not desired (e.g., mammalian cell culture). Such contacting can be performed by including the agent in the media in which the cells, organism or tissue is grown. Contacting in vivo is generally achieved by administration of the agent to a subject which is suspected of being infected by the microbe. One of skill in the art will recognize that an effective amount for in vivo contact may require a higher dose of administration to result in a sufficient amount of target reaching the microbe within the subject's body.

The term “subject” and “individual” are used interchangeably herein, and refer to an animal, for example a human, to whom treatment, including prophylactic treatment, with a composition as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject. As used herein, a “subject” refers to a mammal, preferably a human. The term “individual”, “subject”, and “patient” are used interchangeably.

Administration is performed to promote contact of an effective amount of the administered agent to the microbe within the subject. A therapeutically effective amount of the agent or pharmaceutical composition containing the agent is administered to the subject. The method may further comprise selecting a subject in need of such treatment (e.g., identification of an infected subject. In one embodiment, the agent is administered in combination with or concurrently with one or more other agents that inhibit microbial growth (e.g., those described herein).

Methods of administration include systemic and localized (e.g., topical). Without limitation, these routes include, parenteral administration, and enteral administration.

The route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, and the like. The compounds of the invention can be administered parenterally by injection or by gradual infusion over time and can be delivered by peristaltic means. Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the compounds of the invention are formulated into conventional oral administration forms such as capsules, tablets and tonics.

For topical administration, the pharmaceutical composition (inhibitor of kinase activity) is formulated into ointments, salves, gels, or creams, as is generally known in the art.

The therapeutic compositions of this invention are conventionally administered in the form of a unit dose. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The term “administration” as used herein refers to the presentation of formulations of pharmaceutical compositions described herein, to a subject in a therapeutically effective amount, and includes all routes for dosing or administering drugs or other therapeutics, whether self-administered or administered by medical practitioners. Generally an agent of the present invention is to be administered in the form of a pharmaceutical composition. Pharmaceutical compositions are considered pharmaceutically acceptable for administration to a living organism. For example, they are sterile, the appropriate pH, and ionic strength, for administration. They generally contain the agent formulated in a composition within/in combination with a pharmaceutically acceptable carrier, also known in the art as excipients.

The “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and is compatible with administration to a subject, for example a human.

In one embodiment, the term “therapeutically effective amount” refers to an amount that is sufficient to effect a therapeutically or prophylactically significant reduction in a symptom associated with an infection of a microbe when administered to a typical subject who has the infection. A therapeutically or prophylactically significant reduction in a symptom is, e.g. about 10%, about 20%, about 30%, about 40%, about 50%, about 60%, about 70%, about 80%, about 90%, about 100%, about 125%, about 150% or more as compared to a control or non-treated subject. In many instances, the specific therapeutically effective amount will depend upon many factors, such as the specific microbe and the overall condition of the subject, and will be determined by the skilled practitioner who takes all such relevant factors into consideration. An acceptable benefit/risk ratio will also be considered when determining a therapeutically effective amount. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art, however, that a lower dose or tolerable dose can be administered for medical reasons, psychological reasons or for virtually any other reasons.

In addition, the amount of each component to be administered also depends upon the frequency of administration, such as whether administration is once a day, twice a day, 3 times a day or 4 times a day, once a week; or several times a week, for example 2 or 3, or 4 times a week.

Aspects of the present invention relate to methods for identifying a bVKOR inhibitory agent. One such method is to test one or more test agents for inhibition of bVKOR in a functional assay. Such an assay can be used to screen test agents for the desired activity and specificity towards bVKOR. A test agent that significantly inhibits bVKOR in the assay is thereby identified as an inhibitor of bVKOR. Such an inhibitor is a candidate antimicrobial agent for microbes that express bVKOR. In one embodiment, the bVKOR is obtained from the same microbe which is to be inhibited (e.g. M. tuberculosis). However, it is expected that an agent that inhibits bVKOR from one microbe will significantly inhibit bVKOR from a variety of microbes, and as such will be useful to inhibit microbial growth of such a variety of microbes.

In one embodiment, the test agent is also tested for inhibition of hVKOR in an assay (e.g., an analogous assay), to verify that it does not significantly inhibit hVKOR. Such an assay would be performed, for example, to identify an agent that does not detrimentally inhibit hVKOR when administered to a subject to treat microbial infection. The identified agent can alternatively, or additionally, be tested in an anticoagulation assay.

In one embodiment, the test agent is also tested for inhibition of bVKOR in a second assay for bVKOR activity to further indicate that the test agent is a bVKOR inhibitory agent.

One such functional assay of bVKOR is a disulfide bond formation assay. The assay can be performed in a variety of forms. In one embodiment, the assay is performed in a microbes. One such microbe is E. coli.

In one assay, bVKOR functions as the oxidant of DsbA in the assay, wherein the test agent that significantly inhibits disulfide bond formation in the assay is a bVKOR inhibitory agent. Such an assay can take many different specific forms. One such form is a motility assay, another such form is a β-galactosidase assay, examples of which are both described herein. Another such assay is an alkaline phosphatase assay.

One example of a B-gal assay uses B-gal fused to a bacterial membrane protein (e.g., an E. coli bacterial membrane protein). One such bacterial membrane protein is MalF (e.g., E. coli). Disulfide bond formation assays, such as the ones described herein, can also be adapted to instead have hVKOR in place of bVKOR. Such an assay can be used to screen for agents that inhibit or do not significantly inhibit hVKOR. Such an agent identified by this method can be further screened for anti-coagulant activity by standard methods in the art. In one embodiment, the methods will be used to identify agents that have low anti-coagulant activity.

A test agent that is identified as having significantly more bVKOR inhibitory activity than hVKOR inhibitory activity is a strong candidate for a therapeutic antimicrobial agent against bVKOR containing microbes. Such an agent, thus identified, can be further assayed for growth inhibition activity on a bVKOR expressing microbe, to further validate/identify it as an antimicrobial agent.

Warfarin (Coumadin) is the most widely used oral anticoagulant for prevention and treatment of thrombotic disease but has a narrow therapeutic ratio. It requires regular monitoring of patients as the response to the drug often changes over time, with variations in diet, other medications, etc. and requires readjustment of the dosage based on monitoring of the prothrombin time. This complication may be peculiar to warfarin which binds tightly to albumin whereas only the free (3%) is pharmacologically active. Thus, non-warfarin antagonists of VKOR may be less problematic.

As such, aspects of the present invention also relate to methods of identifying new classes of anticoagulation agents, or for identifying improved anticoagulation agents from modified versions or variants of known anticoagulants (e.g., modified coumadins such as warfarin). By using human VKOR in place of bVKOR, in the screening assays described herein (e.g., the disulfide bond formation assays in E. coli), one can screen test agents for the ability to inhibit human VKOR. Such agents are likely to have anticoagulation activity when administered to a subject in vivo, and are referred to herein as “canadidate anticoagulation agents”. In one embodiment, hVKOR functions as the oxidant of DsbA in the disulfide bond formation assay described herein. The assay can have any useful readout (e.g., motility or β-gal) for inhibition of hVKOR. The ability of the test agent to significantly inhibit disulfide bond formation in the assay indicates that it is a candidate anticoagulation agent. Such an assay can also be used to identify anticoagulation agents that have activity on polymorphism forms of hVKOR which are associated with warfarin resistance (e.g., by using the polymorphic form of hVKOR in the assay).

Once identified, the candidate anticoagulation agent can optionally be further tested in an anti-coagulation assay, to further identify it as an anticoagulation assay.

### Agents and Test Agents

The term “inhibiting” as used herein means that the expression or activity of VKOR protein or variants or homologues thereof, is reduced to an extent, and/or for a time, sufficient to produce the desired effect (e.g. inhibition of disulfide bond formation, antimicrobial activity or anticoagulation activity). The reduction in activity can be due to affecting one or more characteristics of VKOR including decreasing its catalytic activity or by inhibiting a co-factor of VKOR or by binding to VKOR to prevent function (e.g., interaction with another molecule). Inhibition can also be achieved by reducing the overall amount of VKOR present (e.g., by inhibiting gene expression, such as at the translation or transcription level). Inhibition can also be by destabilizing VKOR, leading to increased degradation of the protein.

As used herein, the term “test agent” is used to refer to an agent that is to be tested for a specified activity. Once identified as having that activity, it can then be referred to as an agent with that specified activity.

As used herein, a “test agent” or “agent” can be any purified molecule, substantially purified molecule, molecules that are one or more components of a mixture of compounds, or a mixture of a compound with any other material that can be analyzed using the methods of the present invention. Test agents such as chemicals; small molecules; nucleic acid sequences (e.g., RNAi); nucleic acid analogues; proteins; peptides; aptamers; antibodies; or fragments thereof, can be identified or generated for use in the present invention to inhibit the expression or activity of VKOR (bacterial or human).

Test agents in the form of a protein and/or peptide or fragment thereof can also be designed or identified to inhibit a specific VKOR. Such agents encompass proteins which are normally absent or proteins that are normally endogenously expressed in mammals (e.g. human). Examples of useful proteins are mutated proteins or otherwise modified proteins, fragments of proteins, genetically engineered proteins, genetically modified proteins, peptides, synthetic peptides, recombinant proteins, chimeric proteins, antibodies, midibodies, minibodies, triabodies, humanized proteins, humanized antibodies, chimeric antibodies, modified proteins and fragments thereof. In one embodiment, the agent is a ligand or a portion thereof, or a modified ligand or modified portion thereof. Agents also include antibodies (polyclonal or monoclonal), neutralizing antibodies, antibody fragments, peptides, proteins, peptide-mimetics, aptamers, oligonucleotides, hormones, small molecules, nucleic acids, nucleic acid analogues, carbohydrates or variants thereof that function to inactivate the nucleic acid and/or protein of the gene products identified herein, and those as yet unidentified.

In some embodiments, the agent is a known or unknown compound. It can be from one of numerous chemical classes, such as organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

Test agents can be organic or inorganic chemicals, or biomolecules, and all fragments, analogs, homologs, conjugates, and derivatives thereof. Biomolecules include proteins, polypeptides, nucleic acids, lipids, polysaccharides, and all fragments, analogs, homologs, conjugates, and derivatives thereof. Test agents can be of natural or synthetic origin, and can be isolated or purified from their naturally occurring sources, or can be synthesized de novo. Test agents can be defined in terms of structure or composition, or can be undefined. The agents can be an isolated product of unknown structure, a mixture of several known products, or an undefined composition comprising one or more compounds. Examples of undefined compositions include cell and tissue extracts, growth medium in which prokaryotic, eukaryotic, and archaebacterial cells have been cultured, fermentation broths, protein expression libraries, and the like.

Test agents such as compounds, drugs, and the like are typically organic molecules, preferably small organic compounds having a molecular weight of more than 100 and less than about 10,000 Daltons, preferably, less than about 2000 to 5000 Daltons. In one embodiment, a small molecule has a molecular weight of less than 1000 Daltons, and typically between 300 and 700 Daltons. Test agents may comprise functional groups necessary for structural interaction with proteins, particularly hydrogen bonding, and typically include at least an amine, carbonyl, hydroxyl or carboxyl group, preferably at least two of the functional chemical groups. The candidate or test agents may comprise cyclical carbon or heterocyclic structures, and/or aromatic or polyaromatic structures substituted with one or more of the above functional groups. Candidate or test agents are also found among biomolecules including peptides, saccharides, fatty acids, steroids, purines, pyrimidines, derivatives, structural analogs or combinations thereof.

In one embodiment, the method (e.g., a high throughput screening assay) involves providing a small organic molecule or peptide library of test agents, the library containing a large number of potential VKOR inhibitors. Such “chemical libraries” are then screened in one or more assays, as described herein, to identify those library members (particular chemical species or subclasses) that display a desired characteristic activity. The compounds thus identified can serve as conventional “lead compounds” or can themselves be used as potential or actual products.

In one embodiment, the library of test agents is a combinatorial chemical library. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound) Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14:309-314 (1996) and PCTIUS96/10287), carbohydrate libraries (see, e.g., Liang et al., Science, 274:1520-1522 (1996) and U.S. Pat. No. 5,593,853), small organic molecule libraries (see, e.g., isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.; etc.).

Agents in the form of nucleic acid sequences designed to specifically inhibit gene expression of VKOR are particularly useful. Such a nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

Nucleic acids include, for example but not limited to, DNA, RNA, oligonucleotides, peptide nucleic acid (PNA), pseudo-complementary-PNA (pcPNA), locked nucleic acid (LNA), nucleic acids encoding a protein of interest, RNAi, microRNAi, siRNA, shRNA etc. Inhibitory agents can also be selected from a group of a chemical, small molecule, chemical entity, nucleic acid sequences, nucleic acid analogues or protein or polypeptide or analogue or fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, stRNA, micro RNAi (mRNAi), antisense oligonucleotides etc.

As used herein an “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell.

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi and/or siRNA species but differs in that shRNA species are double stranded hairpin-like structure for increased stability. shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

In one embodiment, siRNA is a double stranded RNA (dsRNA) molecule of about 15, 16, 17, 18, 19, 20, 21, 22, 23, 24, 25, 26, 27, 28, or 30 nucleotides in length, preferably about 15 to about 28 nucleotides, more preferably about 19, 20, 21, 22, 23, 24, or 25 nucleotides in length, and more preferably about 19, 20, 21, 22, or 23 nucleotides in length, and can contain a 3′ and/or 5′ overhang on each strand having a length of about 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In one embodiment of the invention the agent is a catalytic antisense nucleic acid constructs, such as ribozymes, which is capable of cleaving RNA transcripts and thereby preventing the production of the encoded protein. Ribozymes are targeted to and anneal with a particular sequence by virtue of two regions of sequence complementary to the target flanking the ribozyme catalytic site. After binding the ribozyme cleaves the target in a site specific manner. The design and testing of ribozymes which specifically recognize and cleave sequences of the specific gene products is commonly known to persons of ordinary skill in the art.

The agent may result in gene silencing of the target VKOR gene., such as with an RNAi molecule (e.g. siRNA or miRNA). This entails a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the RNAi. In one embodiment, the mRNA levels are decreased by at least about 7%, about 80%, about 90%, about 95%, about 99%, about 100%.

The agent may be applied to the media, where it contacts the cell (such as the progenitor and/or feeder cells) and produces its inhibitory effects. An agent also encompasses any action and/or event the cells are subjected to. The exposure to agent may be continuous or non-continuous.

The agent may function directly in the form in which it is administered. Alternatively, the agent can be modified or utilized intracellularly to produce something which inhibits the VKOR, such as introduction of a nucleic acid sequence into the cell and its transcription resulting in the production of the nucleic acid and/or protein inhibitor of VKOR within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The agent may comprise a vector. Many such vectors useful for transferring exogenous genes into cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The methods described herein can also be used to screen derivatives or variants of molecules with known VKOR inhibitory properties (e.g., phenylpropanoids such as coumarin, warfarin, ferulenol). Such molecules can be chemically modified and the modified to thereby affect their activity, and the resulting molecules screened for the desired VKOR inhibitory activity.

### Compositions

The present invention also relates to isolated nucleic acids which encode the bacterial VKOR protein, such as the bacterial VKOR gene identified in the Examples section herein. The term “isolated” refers to the fact that the nucleic acids are removed or otherwise purified away from the organism in which they naturally occur. They are usually also removed or otherwise purified away from other nucleic acids with which they naturally occur. Also encompassed are modifications of the bacterial VKOR genes so identified (e.g., conservative substitution mutants). The nucleic acid of the invention can be engineered into a vector (e.g., for transport or expression). As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

The nucleic acids within the vectors described herein may be operatively linked to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence. In some examples, transcription of an inserted material is under the control of a promoter sequence (or other transcriptional regulatory sequence) which controls the expression of the recombinant gene in a cell-type in which expression is intended. It will also be understood that the inserted material can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

Also encompassed within the instant invention is a purified and/or isolated expression product of the bacterial VKOR gene, herein referred to as the bacterial VKOR protein. The term purified means that it has been substantially purified away from the bacterial in which it is naturally produced. This can be the result of a purification process, or can be the result of expression of the bacterial VKOR protein from a recombinant vector in another organism.

Also encompassed within the instant invention is an antimicrobial agent and/or an anticoagulation agent identified by the methods described herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (”consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention can be defined in any of the following numbered paragraphs:
1. A method for inhibiting the growth of a microbe that expresses bacterial vitamin K epoxide reductase (bVKOR), comprising contacting the bacterial cell with an effective amount of an agent that inhibits bVKOR.
2. The method of paragraph 1, wherein the agent does not detrimentally inhibit human (h)VKOR.
3. The method of paragraphs 1 or 2, wherein the agent is a drug, ligand or portion thereof, protein, polypeptide, small organic molecule, antisense nucleic acid, RNAi, or antibody.
4. The method of paragraphs 1-3, wherein the agent is a phenylpropanoid, a modified phenylpropanoid, a coumarin or modified coumarin.
5. The method of paragraphs 1-4, wherein the agent is warfarin or a variant thereof or ferulenol or a variant thereof.
6. The method of paragraphs 1-5, wherein the microbe is a microbe identified on FIG. 8 as carrying a bVKOR gene.
7. The method of paragraphs 1-6, wherein the microbe is Mycobacterium tuberculosis.
8. The method of paragraphs 1-7, wherein the agent is identified by the method of paragraph 9 or 15.

9. A method for identifying a bVKOR inhibitory agent, comprising the steps,
a) testing one or more test agents in a disulfide bond formation assay, wherein bVKOR functions as the oxidant of DsbA in the assay; and
b) identifying test agents that significantly inhibit disulfide bond formation in the assay;
   wherein the ability of the candidate agent to significantly inhibit disulfide bond formation in the assay indicates that it is a bVKOR inhibitory agent.
10. The method of paragraph claim 9, wherein the bVKOR is from a microbe identified on FIG. 8 as carrying a bVKOR gene.
11. The method of paragraphs 9-10, wherein the bVKOR is M. tuberculosis.
12. The method of paragraphs 9-11, further comprising testing the test agent identified in step b) in an assay for disulfide bond formation, wherein hVKOR functions as the oxidant of DsbA in the assay, to thereby identify a bVKOR inhibitory agent that does not significantly inhibit hVKOR.
13. The method of paragraphs 9-11, further comprising testing the test agent identified in step b) in a second assay for bVKOR activity to further indicate the test agent is a bVKOR inhibitory agent.
14. The method of paragraph 13, wherein the second assay for bVKOR activity is a growth inhibitory assay for a microbe that naturally expresses bVKOR.
15. A method for identifying an antimicrobial agent, comprising the steps:
   a) assaying test agents for bVKOR inhibitory activity, and for hVKOR inhibitory activity, to thereby identify a test agent that inhibits bVKOR significantly more than it inhibits hVKOR.; and
   b) further assaying the test agent identified in step a) for growth inhibition activity on a bVKOR expressing microbe, wherein an agent that exhibits growth inhibition activity on the microbe, is thereby identified as an antimicrobial agent.
16. The method of paragraph 15, wherein bVKOR inhibitory activity is assayed in a disulfide bond formation assay, wherein bVKOR functions as the oxidant of DsbA in the assay, and wherein hVKOR inhibitory activity is assayed in a disulfide bond formation assay, wherein hVKOR functions as the oxidant of DsbA in the assay.
17. The method of paragraphs 15-16, further comprising assaying the identified test agent of step a) for anti-coagulant activity, wherein a test agent which lacks anti-coagulant activity is further assayed in step b).
18. A method for identifying a candidate anticoagulation agent, comprising the steps:
   a) testing one or more test agents in a disulfide bond formation assay, wherein hVKOR functions as the oxidant of DsbA in the assay; and
   b) identifying test agents that significantly inhibit disulfide bond formation in the assay;
      wherein the ability of the test agent to significantly inhibit disulfide bond formation in the assay indicates that it is a candidate anticoagulation agent.
19. The method of paragraph 18, further comprising testing the identified candidate anticoagulation agents in an anti-coagulation assay, to thereby identify anticoagulation agents.
20. The method of paragraphs 18-19, wherein the hVKOR is wild type hVKOR, or is a polymorphism of hVKOR associated with warfarin resistance.
21. The method of paragraphs 9-20 wherein the test agent is a drug, ligand or portion thereof, protein, polypeptide, small organic molecule, antisense nucleic acid, RNAi, or antibody.
22. The method of paragraphs 9-21, wherein the test agent is a phenylpropanoid, a modified phenylpropanoid, a coumarin or modified coumarin.
23. The method of paragraphs 9-22, wherein the test agent is warfarin or a variant thereof or ferulenol or a variant thereof.
24. The method of paragraphs 9-17, 18-23, wherein the disulfide bond formation assay is a motility assay.
25. The method of paragraphs 9-17, 18-24, wherein the disulfide bond formation assay is a β-gal assay using β-gal fused to a bacterial membrane protein.
26. The method of paragraph 25, wherein the β-gal is fused to bacterial membrane protein MalF, to thereby produce a MalF-β-gal fusion protein.
27. The method of paragraphs 19-17, 18-26, wherein the disulfide bond formation assay is an alkaline phosphatase assay.
28. The method of paragraphs 9-17, 18-27, wherein the disulfide bond formation assay is performed in E. coli.
29. An antimicrobial agent identified by the method of one or more of paragraphs 9-17.
30. An anticoagulation agent identified by the method of one or more of paragraphs 18-28.

The present invention is further illustrated by the following Examples. These Examples are provided to aid in the understanding of the invention and are not construed as a limitation thereof.

### EXAMPLES

### Example 1

### Introduction

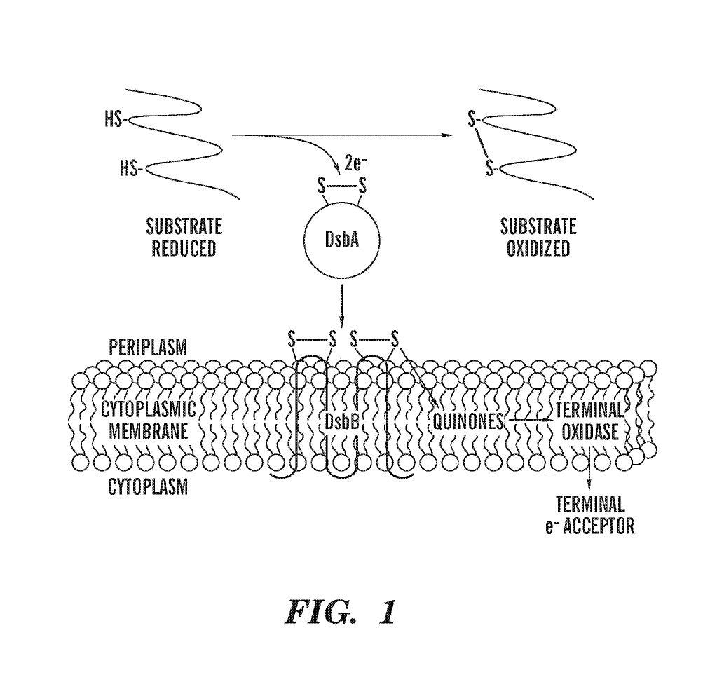

Disulfide bonds, formed by the oxidation of pairs of cysteines, assist folding and stability of many exported proteins. In Escherichia coli, the periplasmic protein DsbA and the membrane-bound protein DsbB promote the introduction of disulfide bonds into proteins (FIG. 1)(1). DsbA, with the active site motif, Cys-X-X-Cys, embedded in a thioredoxin fold, introduces disulfide bonds into proteins that are translocated into the periplasm (2, 3). The active site cysteines of DsbA must be reoxidized for the enzyme to regain activity, a step catalyzed by DsbB(4). DsbB then shuttles electrons received from DsbA to the electron transport chain via membrane-bound quinones (5, 6).

Oxidative protein folding has been studied extensively only in a small fraction of bacterial species. Given the considerable biological diversity within the domain Bacteria, a more extensive analysis of this group of organisms may reveal novel aspects of disulfide bond formation. The availability of hundreds of complete bacterial genome sequences permits a broad bioinformatic analysis.

For most organisms, disulfide-bonded proteins are restricted to non-cytoplasmic compartments. However, Mallick et al found that cytoplasmic proteins from some hyperthermophilic archaea contain disulfide bonds (7). Further, they showed that the presence of disulfide-bonded proteins in the cytoplasm correlates with a bias for even numbers of cysteines in the archaeal proteome. One explanation for an enrichment of even numbers of cysteines in proteins with disulfide bonds is that odd numbers of cysteines in a protein could allow the formation of inappropriate disulfide bonds, resulting in a misfolded protein (8). In fact, organisms from bacteria to eukaryotes express disulfide bond isomerases that ensure the correct array of disulfide bonds in a protein after such “mistakes” are made (9, 10). To avoid the problem of mis-matched cysteines, there may be evolutionary pressure to select for an even number of cysteines in proteins with disulfide bonds.

It was reasoned that a bioinformatic analysis to determine whether proteins in the cell envelope of different bacteria have significant biases for even numbers of cysteines could indicate whether this compartment contains disulfide-bonded proteins. Here it is shown that this is the case for E. coli. The cysteine content of predicted cell envelope proteins from each of 375 other bacterial genomes was then analyzed to assess whether each of these organisms may have disulfide-bonded proteins. Homology searches in each genome to identify members of the DsbA and DsbB protein families were also used. The merging of these data enabled the generation of predictions as to whether oxidative folding is likely to occur in the cell envelope of each of the bacteria examined, and, if so, whether the organism uses the Dsb pathway.

The obtained results lead to the proposal that oxidative folding of cell envelope proteins may not be a well-conserved feature of bacterial cell biology. To support this hypothesis, experimental data from Bacteroides fragilis NCTC9343 is presented herein. In addition, many bacteria were predicted by this analysis to carry out disulfide bond formation, but lack a homolog of DsbB. This observation has led to the identification of a candidate for a novel disulfide bond formation enzyme, the bacterial homolog of the mammalian enzyme vitamin K epoxide reductase (VKOR). Experimental evidence is presented for a DsbB-like activity of the Mycobacterium tuberculosis H37Rv homolog of VKOR. This enzyme may play a role analogous to DsbB in several major bacterial phyla.

### Results

Cysteine Composition of Cell Envelope Proteins in E. coli

The E. coli proteome was examined to determine whether differences in patterns of cysteine distribution correlate with the compartment in which disulfide bond formation takes place, the cell envelope. The proteome was divided into 5 classes, based on subcellular location that was predicted by bioinformatic approaches for analyzing the open reading frames in the genome (see Methods). These protein classes are: 1)cytoplasmic, 2)membrane spanning segments of transmembrane proteins (TM-membrane spanning), 3)cytoplasmic loops and domains of transmembrane proteins (TM-cytoplasmic), 4)periplasmic loops and domains of transmembrane proteins (TM-periplasmic), and 5) “exported” proteins, which we define as the mature parts of signal-sequence directed exported proteins, which includes most periplasmic, outer membrane-bound, and extracellular proteins.

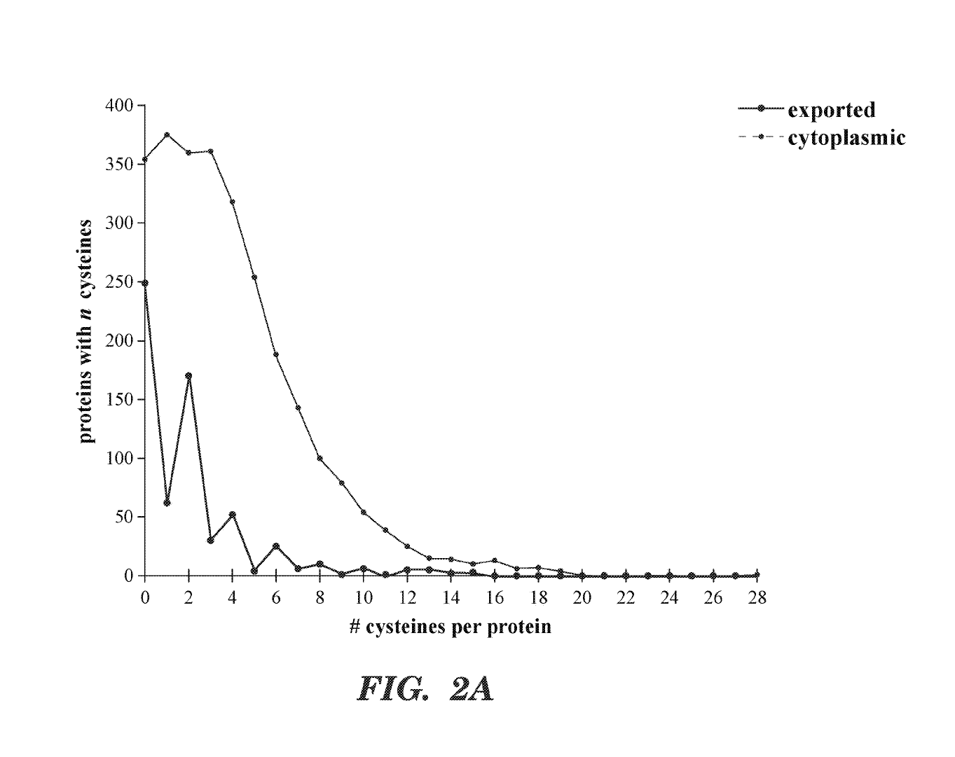

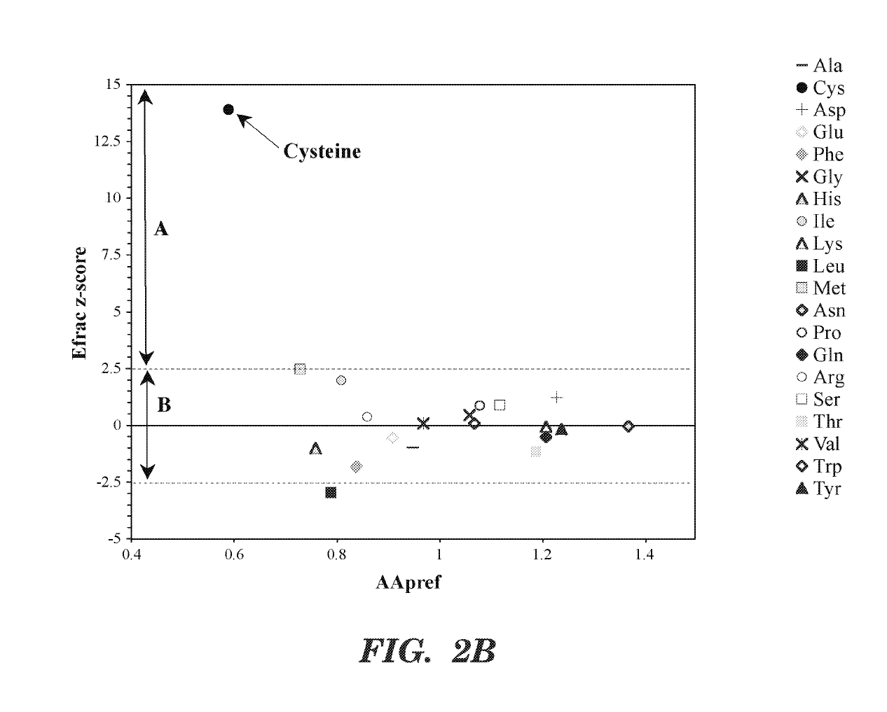

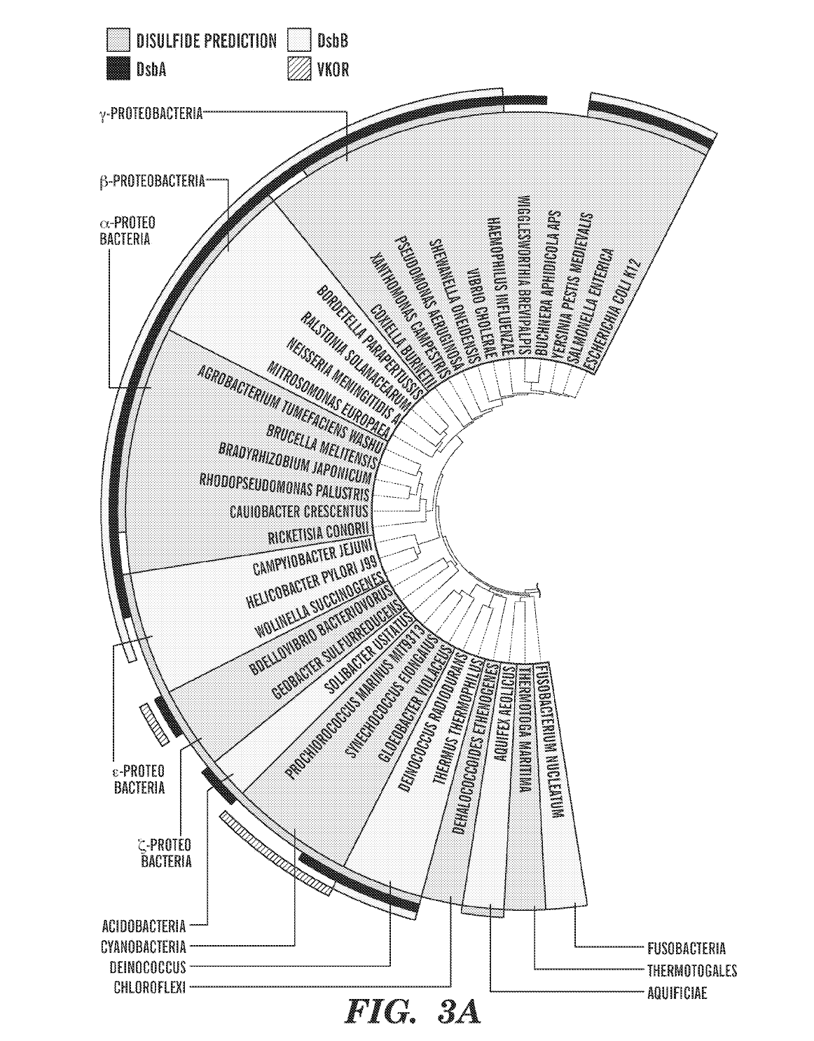

All proteins from compartments 1-5 were analyzed both for the bias for even numbers of cysteines and for overall cysteine content. First, the percentage of cysteines in exported proteins (class 5) was found to be considerably smaller than the percentage of cysteines in the entire proteome; 39% of exported proteins have cysteine as compared with 87% of cytoplasmic proteins. This strong bias against cysteine in exported proteins has been noted before in the analysis of a much smaller subset of proteins(11). Second, a substantial majority of cysteine-containing exported proteins (71%) have even numbers of cysteines, reflected in the sawtooth shape of the plot in FIG. 2A.

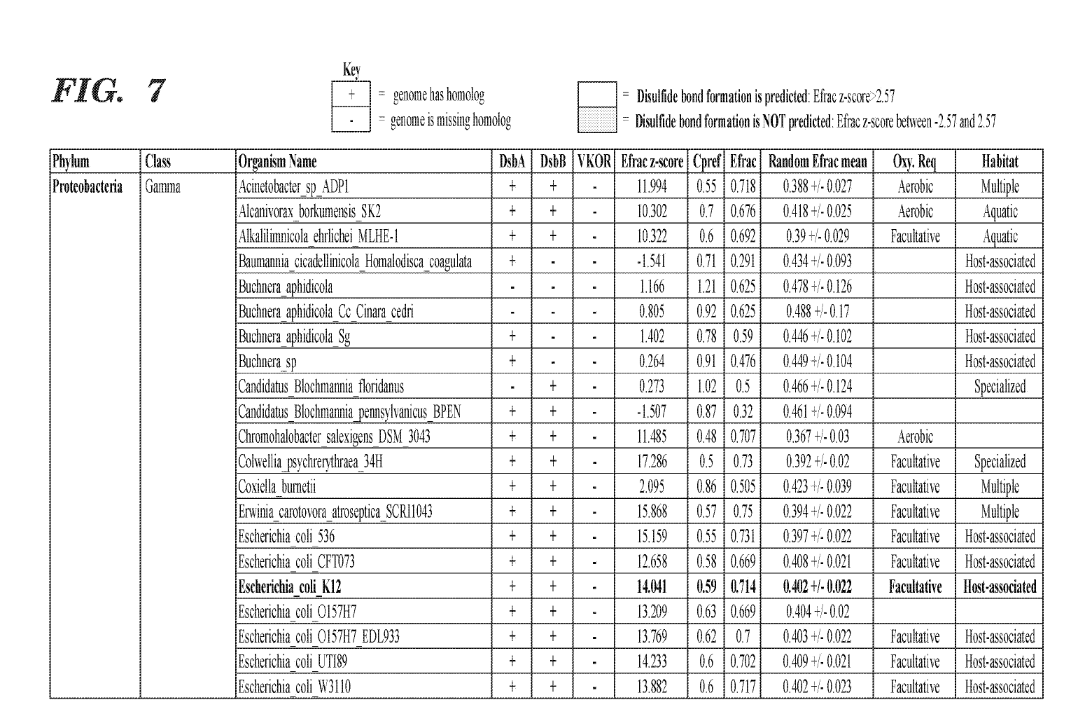

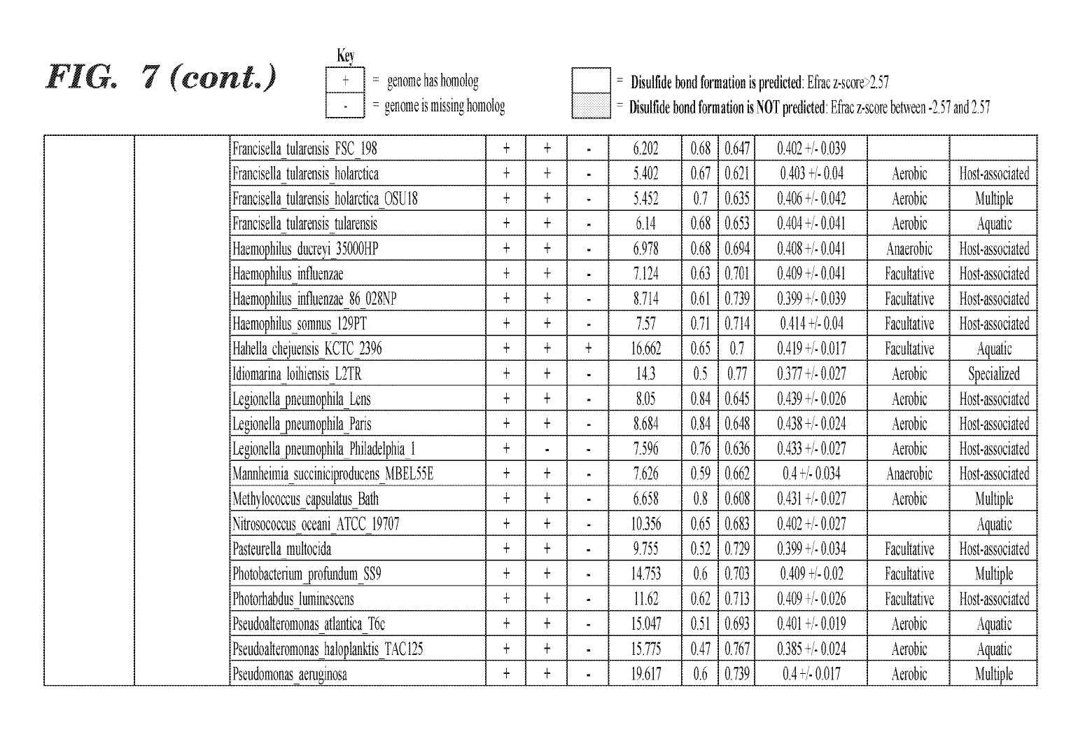

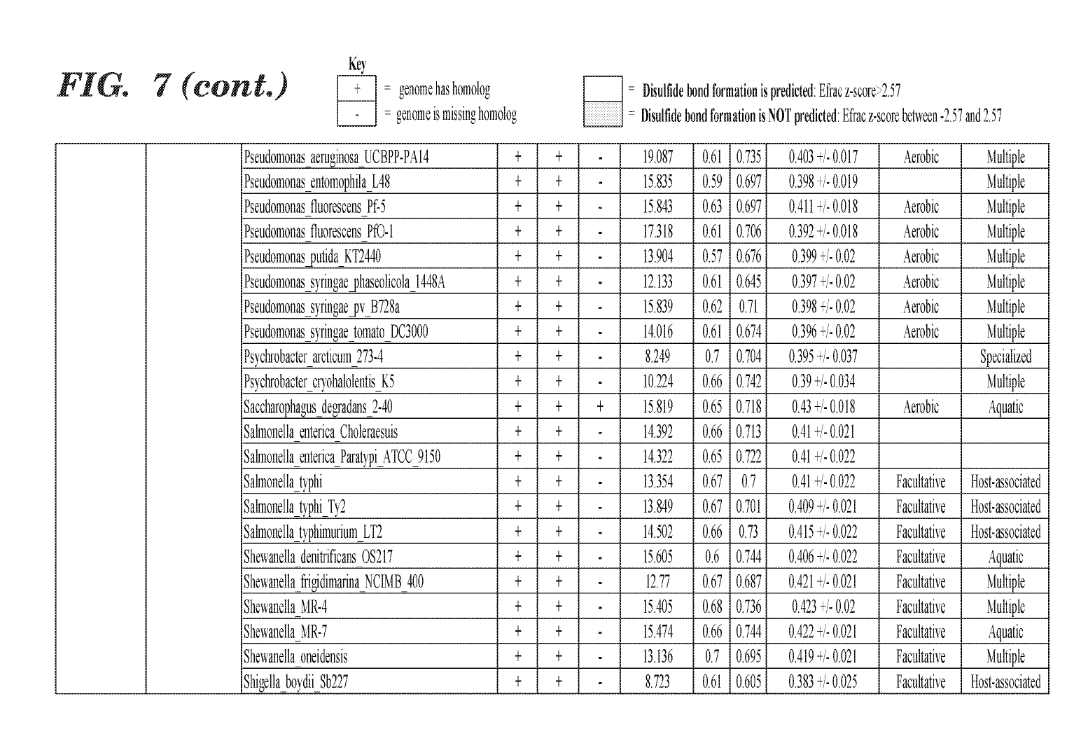

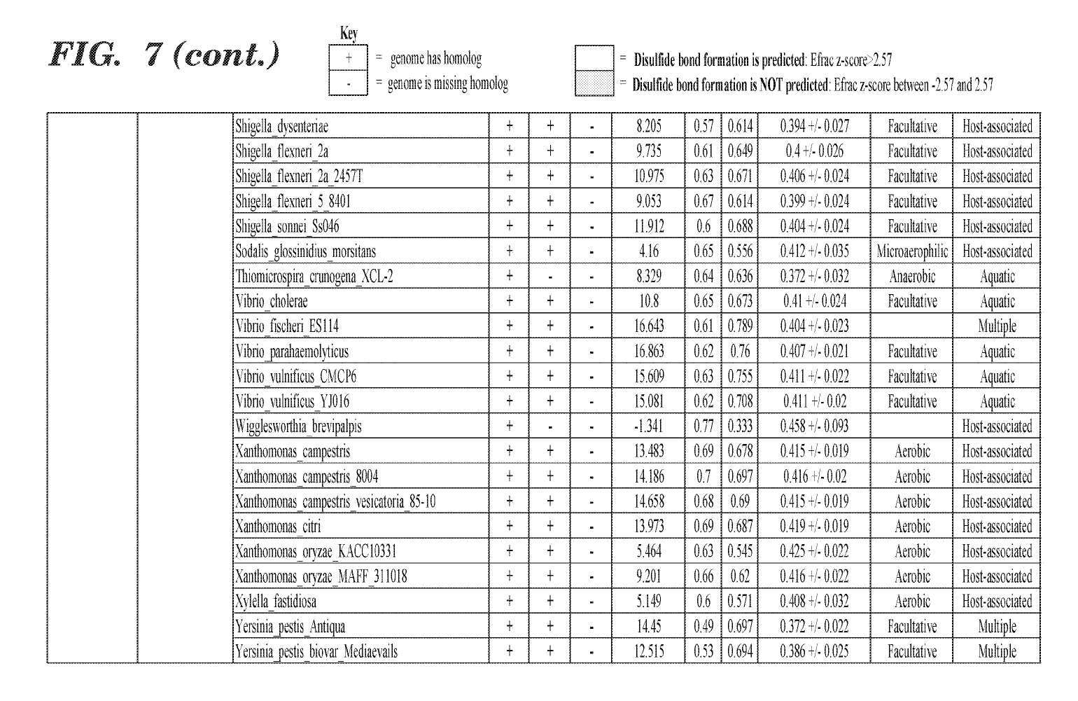

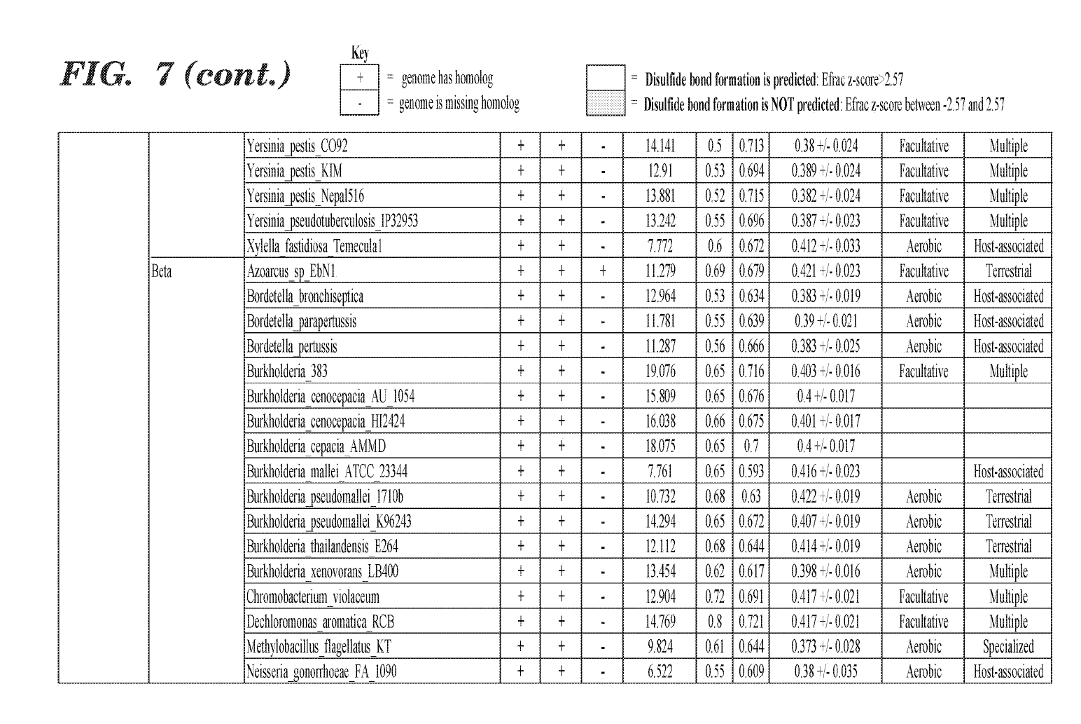

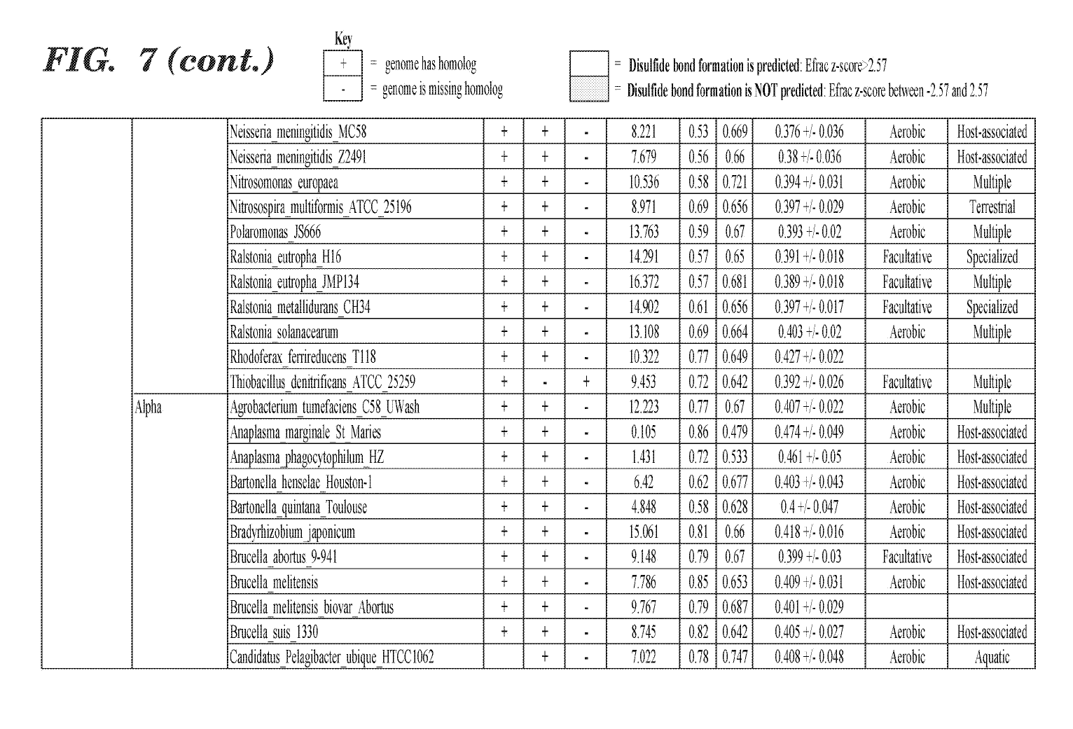

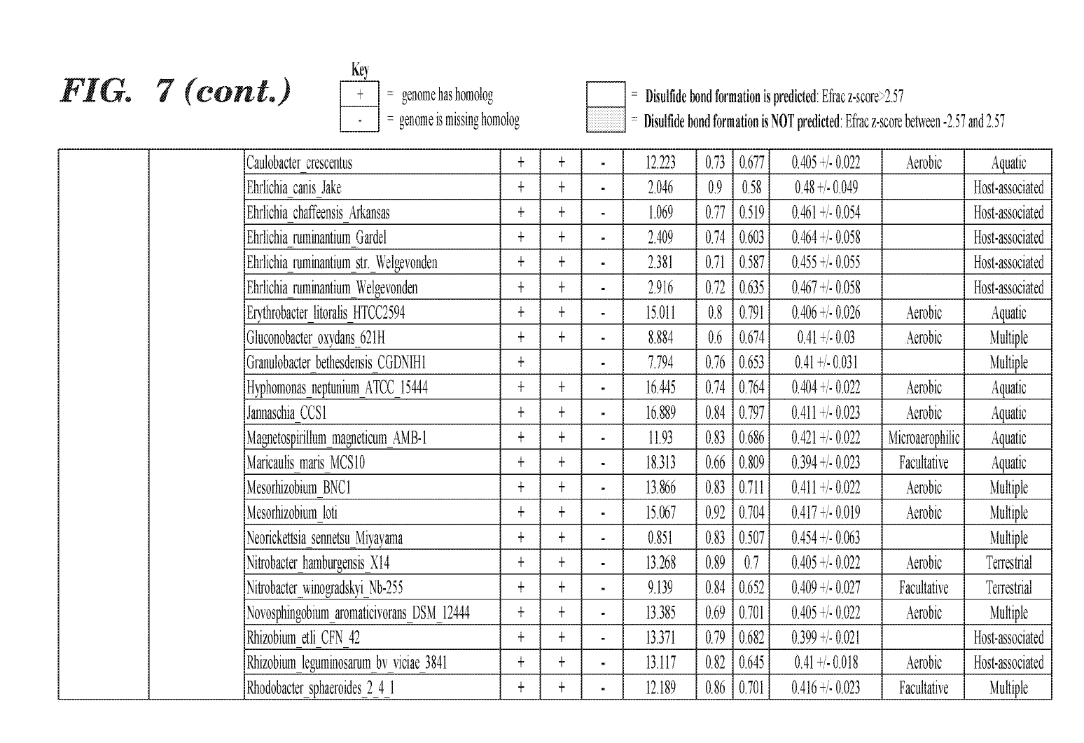

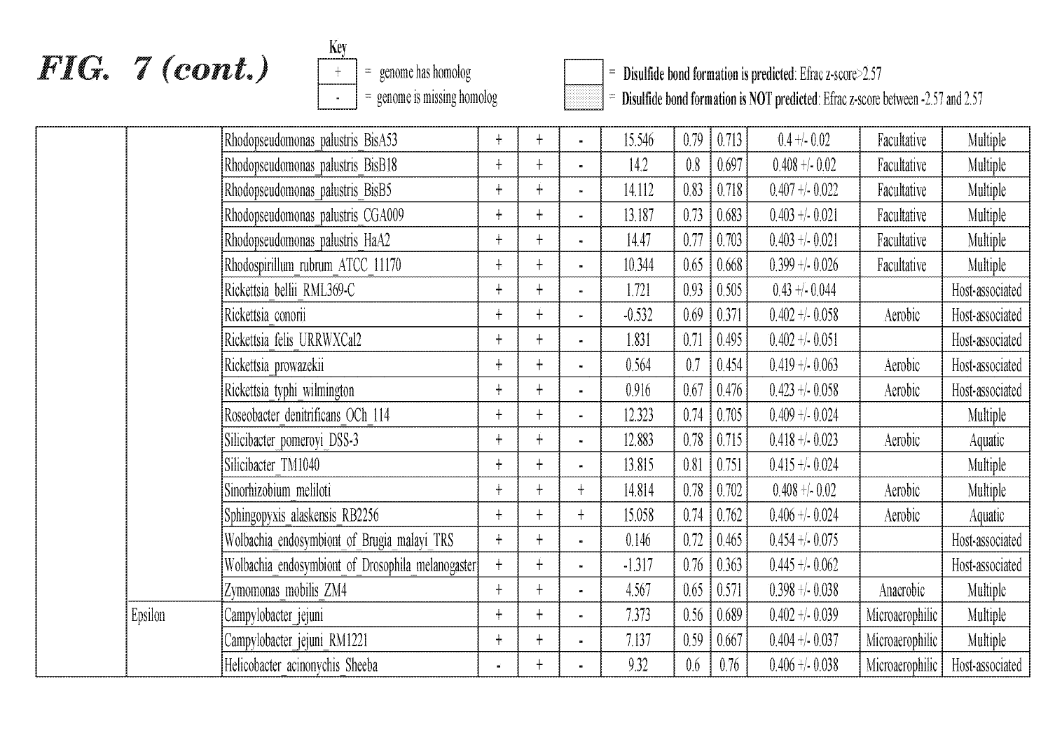

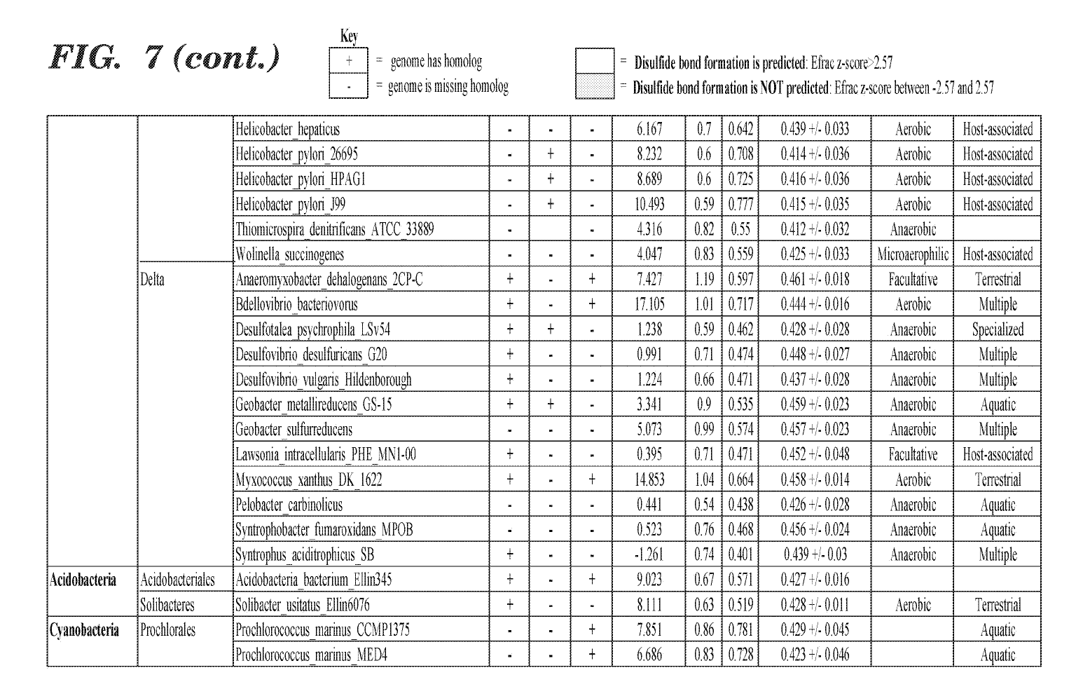

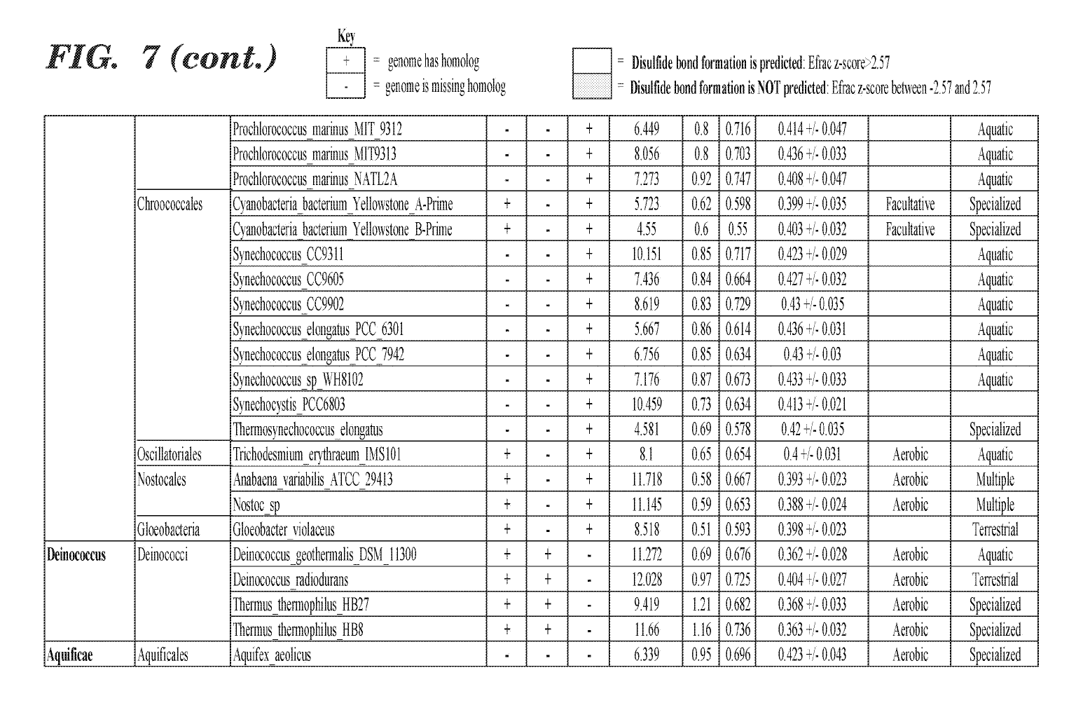

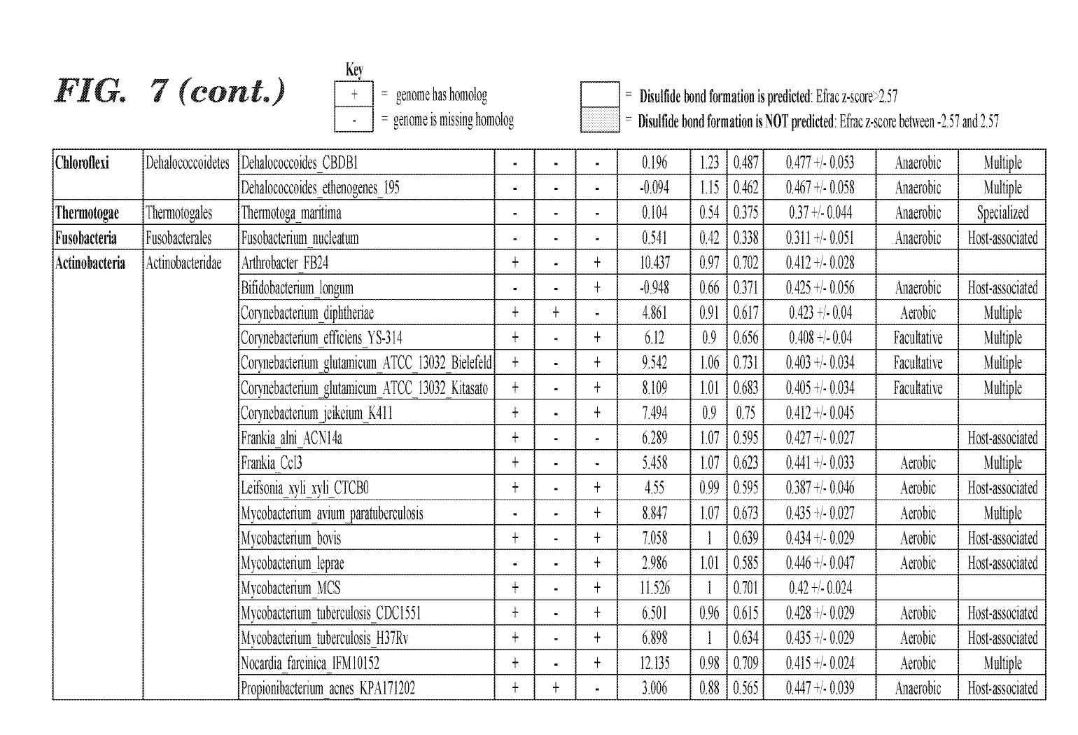

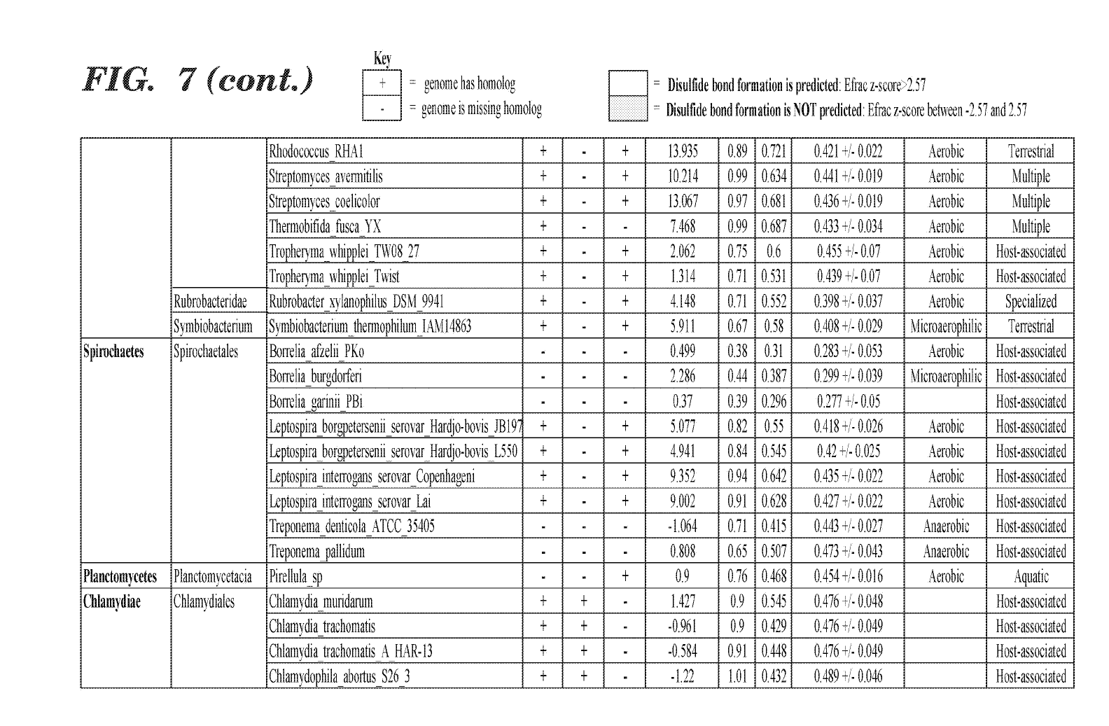

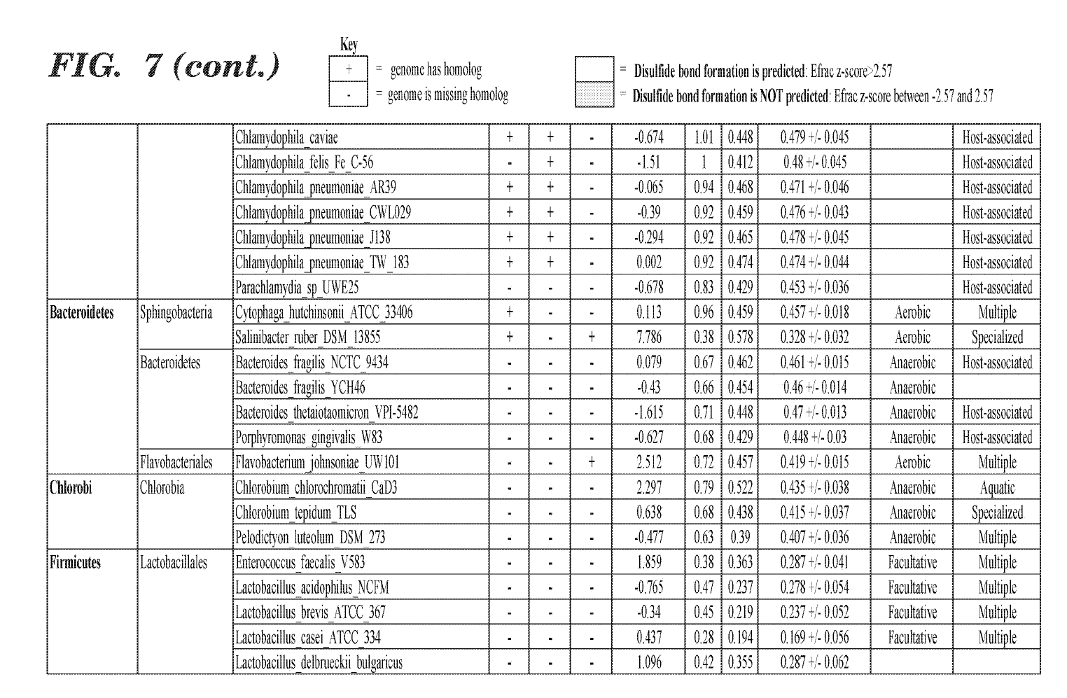

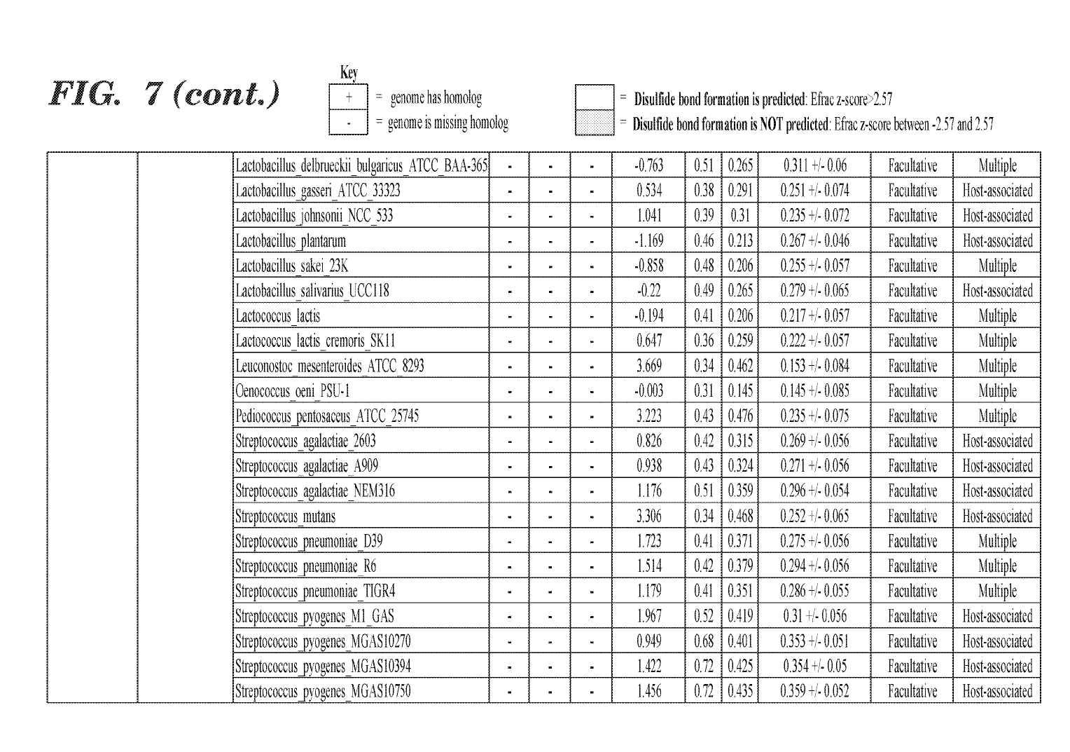

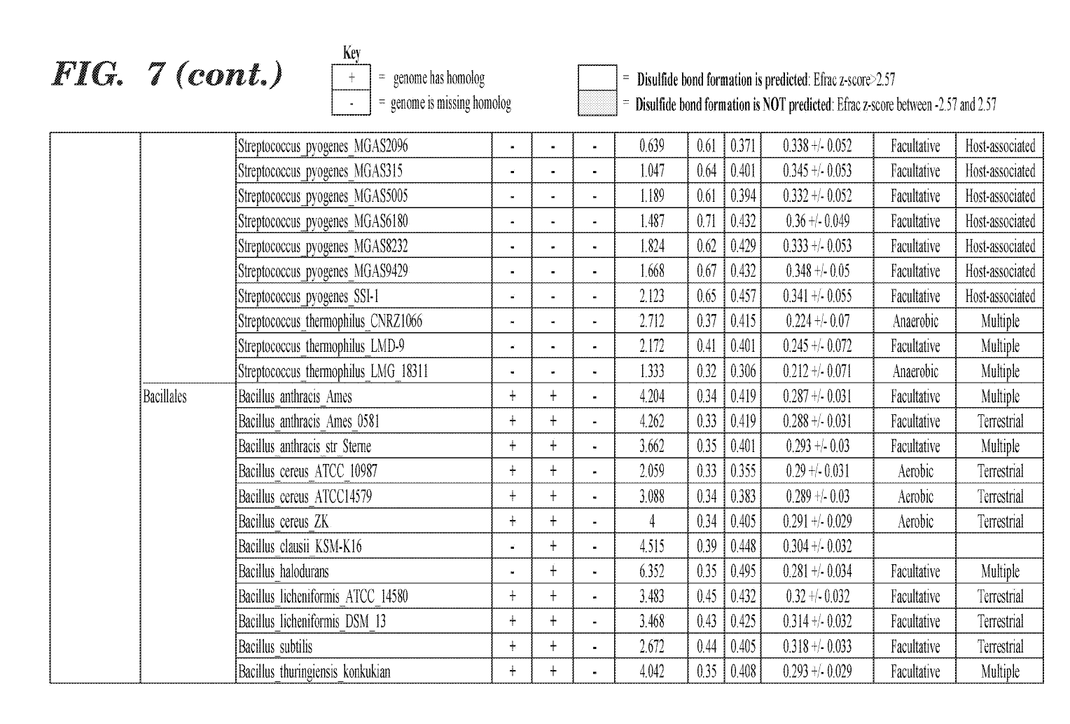

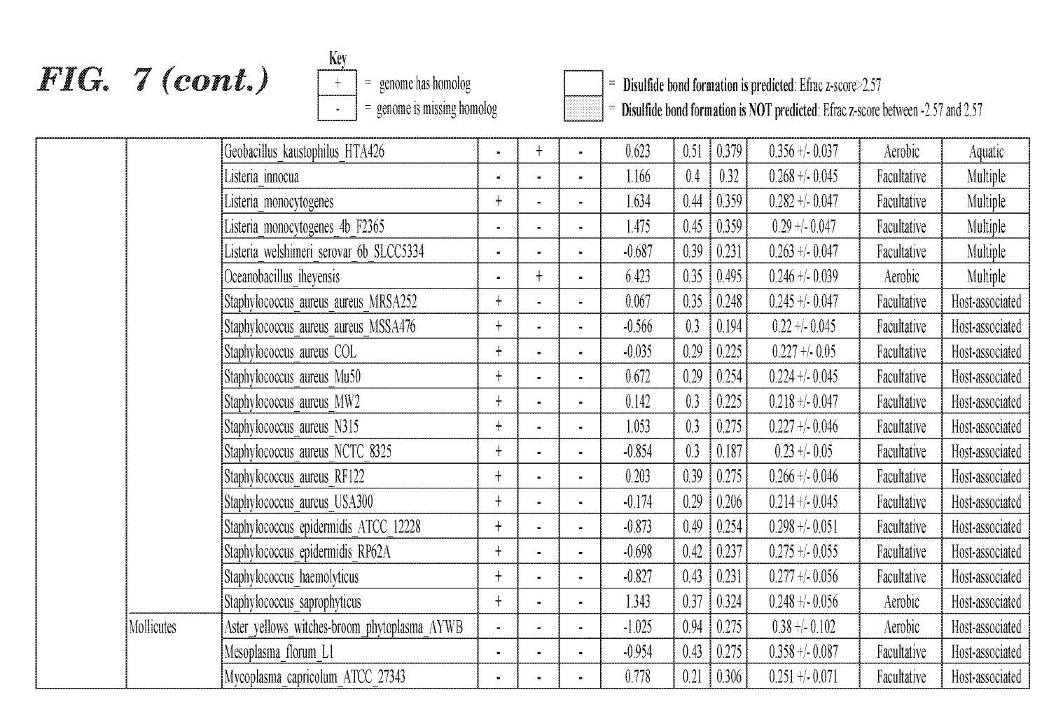

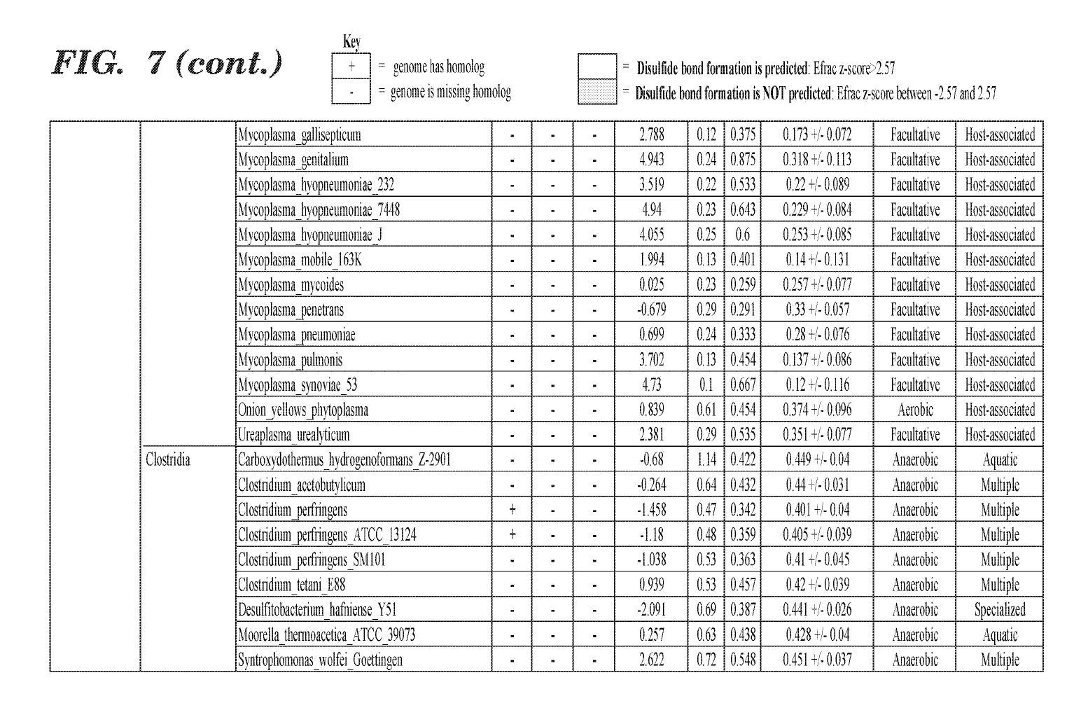

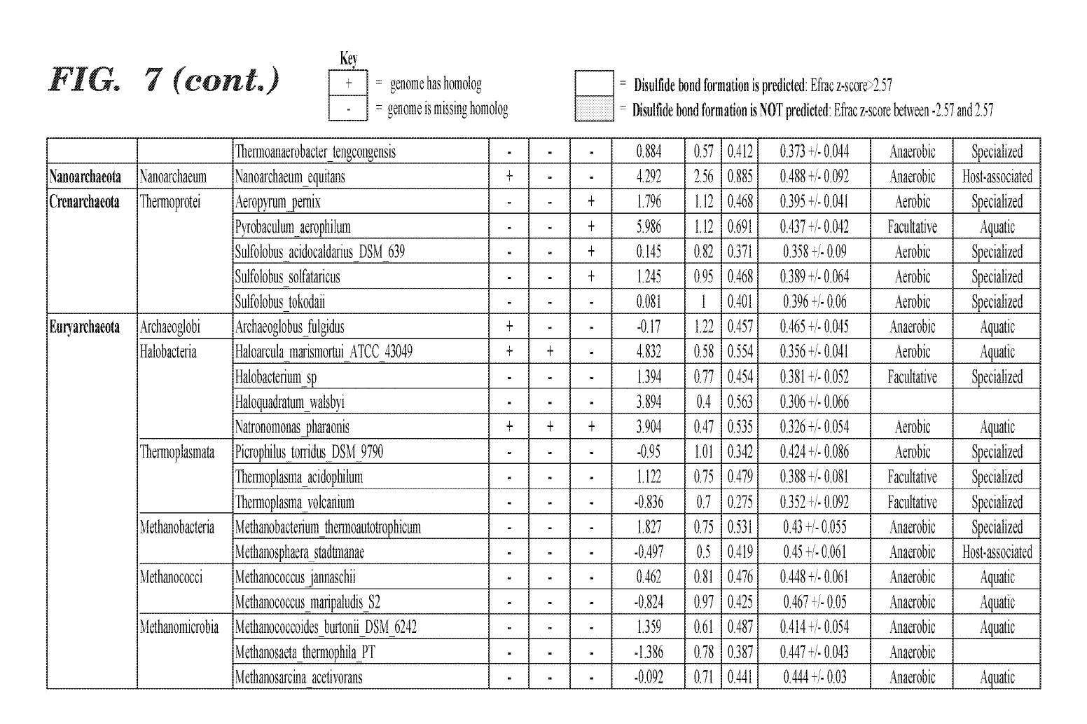

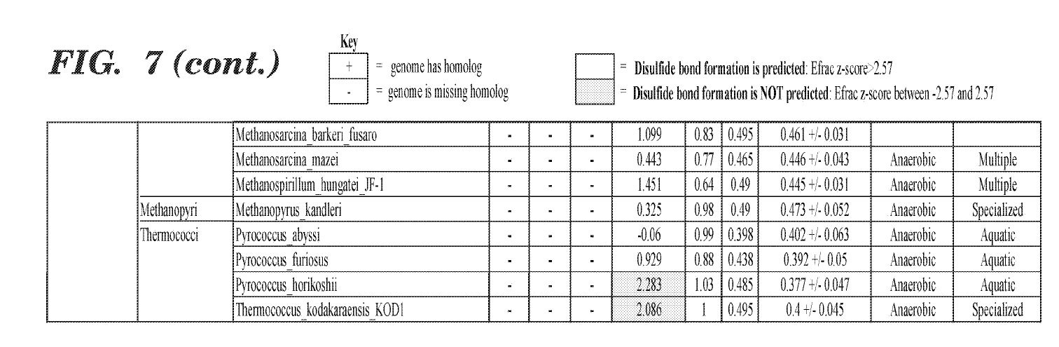

To determine the significance of this latter finding, predictions of a null hypothesis in which cysteines are distributed randomly among open reading frames for exported proteins were tested. This test takes into account the cysteine composition of the compartment, as well as the length of each protein within the compartment, and then distributes the cysteines at random within each protein (according to a Poisson distribution, see Methods). For each class of proteins, the actual fraction of proteins were compared with even numbers of cysteines to the fraction predicted by the random model (FIG. 7). The fraction of proteins predicted by the random model to have an even number of cysteines is different for each class because the amino acid composition of each class is different and the distribution of total number of residues per protein varies in different classes.

While proteins or portions of proteins that are predicted to be localized to or pass through the periplasm have a highly significant bias for even numbers of cysteines (classes 4 and 5, FIG. 7), membrane-spanning segments, portions of membrane proteins predicted to be exposed to the cytoplasmic compartment, as well as soluble cytoplasmic proteins do not (classes 1,2,3, FIG. 7). Strikingly, this analysis shows for E. coli that cysteine distributed according to the random model would result in 40.2% (mean value) of exported cysteine-containing proteins having an even number of cysteines, in contrast to the 71% actually observed, a latter number 14 standard deviations above the mean of the random model value. The bias for (ratio of observed to expected) even numbers of cysteines in both the mature exported (class 5) and TM-periplasmic (class 4) classes is similar (FIG. 7). Their z-scores are different because the standard deviation of the TM-periplasmic class is larger, a consequence of the smaller number of residues per protein in the TM-periplasmic class. For this reason the exported class of proteins, class 5, for all of subsequent analyses was used.

The data for other amino acids in exported proteins was compared to see if these features are unique to cysteine. As done with cysteine, for each of the other amino acids, the fraction of exported proteins with even numbers of that amino acid were calculated, as well as the predicted fraction of proteins containing an even number of cysteines, according to the random model of amino acid distribution, as described above. The number of standard deviations of the actual data from the mean of the random calculation gave a z-score (FIG. 2B). The Z-score is plotted against the AApref, a measure of the preference for or against incorporating the amino acid in an exported protein. AApref is the ratio of the frequency of the amino acid in the class to the frequency in the proteins encoded in the entire genome. Of all the amino acids, only cysteine shows a strong bias against incorporation into exported proteins. For all other amino acids, the fraction of exported proteins with an even number of the amino acid is approximately what would be expected from the random model.

Based on previous results of Mallick et al.(7), it was suspected that these values for cysteine in exported proteins that differentiate it from other amino acids reflect the active formation of disulfide bonds in the E. coli K12 periplasm. Therefore, a preference for even numbers of cysteines in exported proteins may be an indicator of disulfide bonds in the cell envelope proteins of a given organism.

### Computational Analysis of Disulfide Bond Formation in Other Sequenced Bacterial Genomes

This analysis of cysteine patterns in exported proteins was extended to 375 fully sequenced bacterial genomes. As with E. coli, the subcellular localization of all proteins for each genome was predicted and then the fraction of cysteine-containing exported proteins with even numbers of cysteines, as well as the fraction if cysteine were distributed according to the random model in that set of proteins, was calculated. The number of standard deviations of the actual data from the mean obtained with the random model gave a z-score for each genome which is then plotted versus the bias against incorporating cysteine into exported proteins (Cpref) for that genome.

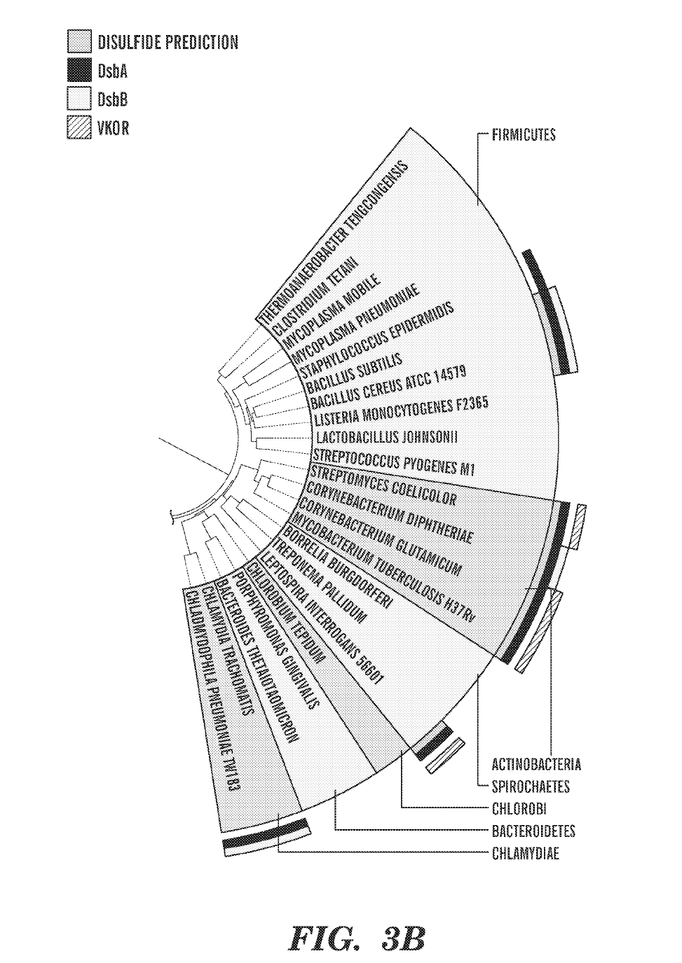

Bacteria were classified according to whether their exported proteins showed a significant bias for even numbers of cysteine: It was hypothesized that 1) bacteria that oxidatively fold cell envelope proteins should have significantly more even numbers of cysteines in exported proteins than is predicted by our random model (z-score above the 99% confidence level, 2.57, and 2) bacteria that do not generally make disulfide bonds would lack significant bias for even number of cysteines in exported proteins (z-score within the 99% confidence level, 2.57>z>-2.57. These data were then combined with the results of homology searches for members of the DsbA and DsbB family of proteins to allow further deductions about the biology of disulfide bond formation in each bacterium (representative dataset in FIG. 3, for complete dataset, see FIG. 7).

### Bacteria Predicted to Catalyze Disulfide Bond Formation in Cell Envelope Proteins

It was found that most bacteria belonging to the same phylum as E. coli, the Proteobacteria, have DsbA and DsbB homologs, as has been observed previously (12, 13), as well as similarly high fractions of exported proteins with even numbers of cysteines and low cysteine content. These results suggest that a similar thiol redox biology of cysteine-containing proteins in the cell envelope of most of these organisms. Exceptions include the obligate intracellular proteobacteria, the obligate anaerobic proteobacteria, and the delta-proteobacteria. Surprisingly, there was only one other group of bacteria (members of the Phylum Deinococcus-Thermus) showing high fractions of exported proteins with even numbers of cysteines and homologs of both DsbA and DsbB. All other major groups of bacteria examined differed from the Proteobacteria either in that they lacked a complete DsbA/B pathway or in the bias for even numbers of cysteines in exported proteins.

One striking pattern that emerges from this analysis is that no homolog of DsbB is found in a large number of bacteria that we predicted should catalyze disulfide bond formation and which do have a DsbA homolog. This group includes most Actinobacteria, Cyanobacteria (including chloroplasts), aerobic delta-proteobacteria, Spirochaetes in the genus Leptospira, and the Bacteroidete Salinibacter ruber. The bioinformatic prediction of disulfide bond formation for some of these organisms is consistent with in vitro and in vivo studies that directly identified disulfide bond-containing exported proteins (14-16). Thus, these organisms would need an alternative to DsbB for the reoxidation of DsbA.

In the genomes of Salinibacter ruber, Leptospira interrogans and the delta-proteobacterium, Bdellovibrio bacteriovorus, the DsbA homolog is fused to an integral membrane protein—a homolog of the eukaryotic enzyme vitamin K epoxide reductase (VKOR). VKOR has recently been characterized in mammals (17, 18), but the function of bacterial homologs of VKOR is unknown. The activity of mammalian VKOR serves to maintain the cellular pool of vitamin K, a quinone, in the reduced state. In mammals, reduced vitamin K (as opposed to the oxidized form, vitamin K 2,3 epoxide) is required as a cofactor for the gamma-carboxylation of glutamic acid residues in the blood clotting protein prothrombin. VKOR has four highly conserved cysteine residues, two of which are in a Cys-X-X-Cys motif and are essential for catalytic activity in vitro (19, 20). Recent work suggests that eukaryotic VKOR activity is stimulated by the presence of protein disulfide isomerase (PDI)(21), which is the thioredoxin-like protein that catalyzes disulfide bond formation in eukaryotes (22). This passage of electrons from PDI to VKOR to a quinone are analogous to the passage of electrons from DsbA to quinones via DsbB in E. coli during disulfide bond formation. The herein reported genomic analysis indicated that most of the Actinobacteria, Cyanobacteria, delta-proteobacteria, and Spirochaetes that are missing a DsbB homolog, have a VKOR homolog (FIG. 3, and FIG. 7), that the VKOR gene in bacteria is mostly limited to these genomes, and thus, that its distribution correlates with the absence of DsbB in organisms predicted to carry out disulfide bond formation. Examples where a VKOR homolog was fused to either a DsbA homolog or a thioredoxin homolog in bacteria that were missing DsbB as noted before, were found (20).

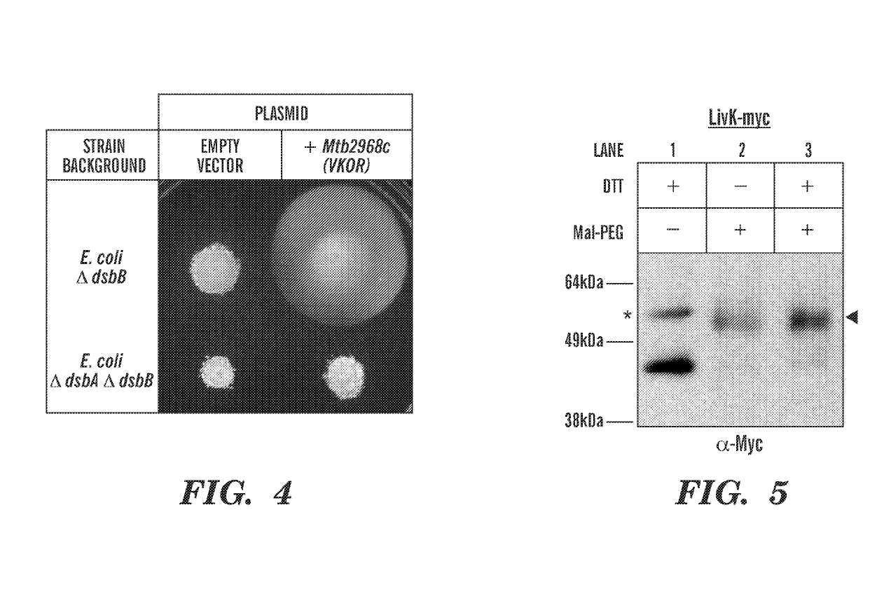

It was hypothesized that the bacterial VKOR homolog serves the role of DsbB in these bacteria. As a preliminary test of this hypothesis, one of the bacterial homologs of VKOR, from Mycobacterium tuberculosis H37Rv, was cloned and tested for its ability to complement a strain of E. coli K12 deleted for dsbB. A motility assay was used to assess complementation for disulfide bond formation since one of the flagellar structural proteins, FlgI, requires a disulfide bond for stability. The M. tuberculosis VKOR homolog complements the motility defect of ΔdsbB strain, albeit not to wild type levels (FIG. 4). This complementation is dependent on the presence of dsbA, indicating that VKOR is restoring disulfide bond formation to FlgI, not by acting as a general oxidant, but rather through the intermediary of DsbA, as does DsbB. Thus, the bacterial VKOR may be an enzyme that plays a role analogous to DsbB in several major phyla of bacteria.

Bacteria that are Predicted to have Limited Disulfide Bond Formation

The data suggests that several groups of bacteria have no or very few proteins with disulfide bonds based on their low fractions of exported proteins with even number of cysteines. These bacteria comprise a phylogenetically diverse set of organisms, with species from the phyla Proteobacteria, Actinobacteria, Bacteroidetes, Firmicutes, and Spirochaetes as well as all sequenced species from the phyla Chlorobi, Fusobacterium, Thermotogae, and Chloroflexi. Many of these bacteria also lack homologs of DsbA and DsbB, consistent with the prediction that they do not oxidatively fold exported proteins. Thus, a potentially novel type of cell envelope biology may be present in this group of organisms, since the bacterial cell envelope is generally thought of as an oxidizing environment.

A preliminary test of the hypothesis that these organisms do not have an oxidizing cell envelope was performed using a bacterium Bacteroides fragilis predicted by our analysis to fall into this class. To assess disulfide bond formation in this organism, an E. coli K12 protein known to have a disulfide bond, LivK (23) was cloned and expressed in B. fragilis. Cysteine alkylation experiments were used to determine if the protein acquired a disulfide bond when expressed in B. fragilis. If the cysteines of LivK are free (not disulfide-bonded), they will react with the alkylating agent, resulting in an increase in molecular weight. When expressed in B. fragilis, LivK is exported (data not shown), and the cysteines of the protein are alkylated, indicating the absence of disulfide bonds in the protein (FIG. 5). E. coli alkaline phosphatase was also expressed in B. fragilis from the same vector, but the protein was not detected, suggesting that the protein may have been degraded due to a lack of disulfide bonds or was not well-expressed. Thus, unlike E. coli DsbA, which forms disulfide bonds in a wide range of substrates from both eukaryotes and bacteria without any apparent specificity towards different substrates, B. fragilis may either have a very limited ability to make disulfide bonds or lack such a system altogether. This preliminary finding is consistent with the bioinformatic data prediction for B. fragilis and stands in contrast to the E. coli cell envelope, generally thought of as an oxidizing environment.

Although the bacteria that were predicted lack protein disulfide bonds are phylogenetically diverse, a common trait of many of them is their classification as obligate anaerobes or obligately intracellular organisms. This observation is striking considering that, in some cases, the closest relatives of these bacteria are aerobic or free-living bacteria that are predicted to have an oxidizing envelope. The indication that these groups of functionally, but not necessarily phylogenetically, related bacteria may lack disulfide bond formation suggests that they may share some common environmental and/or genetic influences. For instance, the generally reducing environments (e.g. anaerobic sediments or host cell cytoplasm) that these organisms inhabit may be unfavorable for disulfide bond formation. In addition, a number of the obligate anaerobes are obligate fermenting organisms, including members of the genera Clostridia and Lactobacillales, within the phylum Firmicutes. These bacteria are generally thought to lack an electron transport chain (24). Since disulfide bond formation in E. coli is linked to the electron transport chain, an obligately fermentative metabolism may be incompatible with the ability to form disulfide bonds.

### Dsb Homologs in Genomes Predicted to Lack Disulfide Bond Formation

The combination of cysteine counting and analysis of genomes for DsbA/DsbB homologs did not always allow clear predictions of the general redox state of the cysteine-containing proteins in the cell envelope. For example, Bacillus subtilis has slightly more even numbers of cysteines in exported proteins than is predicted by the random model (z=2.67). Yet, systems (Bdb proteins) for disulfide bond formation in exported proteins have been described in this organism that are related by sequence homology to DsbA/DsbB. Furthermore, disulfide bonds in at least two B. subtilis proteins, sublancin (a peptide antibiotic with two disulfide bonds) and the competence protein ComCG, depend on these systems (25, 26). However, no other native substrates of these disulfide bond formation pathways are known. It remains to be determined the extent to which B. subtilis and other Firmicutes with the Bdb system catalyze disulfide bond formation.

It is also predicted that the members of the phylum Chlamydiales do not generally make disulfide bonds, yet these bacteria all have DsbA and DsbB homologs, and have at least one disulfide-bonded protein, the major outer membrane protein (27). However, a recent paper reported in vitro evidence that an exported thioredoxin-like protein from Chlamydia pnuemoniae, DsbH, has reducing activity, suggesting that this organism may actively reduce proteins in the cell envelope (28).

The delta-proteobacterium Geobacter metallireducens also has a low number of exported proteins with even numbers of cysteines and yet has DsbA/DsbB homologs encoded in its genome. The dsbA and dsbB of this bacterium are linked in a putative operon, rather than located at different chromosomal sites as they are in E. coli K12 and most other organisms. This putative operon is occasionally found as an additional copy of dsbA/B in some close relatives of E. coli K12, including pathogenic E. coli (CFT073, UTI89, APEC 01, 536), and some Salmonella species. All organisms with this conserved dsbAB putative operon also include a tightly linked gene astA, encoding the secreted enzyme arylsulfotransferase, an enzyme which in Enterobacter sakazakii has a disulfide bond essential for its activity (29). It may be that this DsbA/B is present in organisms such as G. metallireducens specifically to act on arylsulfotransferase or a small subset of proteins.

Since, for each of these genomes, we find a conflict between the cysteine counting data and the presence of DsbA and DsbB homologs, the balance between reduced and oxidized proteins in the cell envelope of these bacteria may be a complex issue. Surprisingly, examination of the genomic context of the DsbA and DsbB homologs in all of these genomes shows that the putative dsbA/dsbB operon, in some cases, is found on a prophage or plasmids. This observation may be of interest from an evolutionary perspective, since it suggests that DsbA and DsbB homologs found outside of Proteobacteria may have moved into these genomes via horizontal gene transfer.

### Discussion

A multi-faceted bioinformatic approach has been used to generate predictions regarding the capacity of different bacteria to catalyze disulfide bond formation. This work suggests that there is considerable diversity in mechanism and capacity for disulfide bond formation across bacterial species.

This analysis led to the identification of a protein, a homolog of mammalian enzyme vitamin K epoxide reductase (VKOR), which appears to be an alternative to DsbB in several major phyla of bacteria. This protein is shown to restore disulfide bond formation to E. coli deleted for dsbB in a DsbA-dependent manner. Although DsbB and VKOR do not show obvious similarities at the sequence level, they do appear to be similar in the reactions they perform, that is, the passage of electrons from thioredoxin-like proteins to membrane bound quinones via redox-active cysteines in the protein. Since the mammalian VKOR is of considerable medical interest due to its role in blood clotting in humans, these studies of the bacterial homolog could provide insights into the biological properties and cellular roles of this protein family. Further work will assess the dependence of M. tuberculosis on VKOR for disulfide bond formation and the mechanistic similarities between the bacterial VKOR and DsbB. During the preparation of this manuscript, a similar publication reaching a similar conclusion about the role of the VKOR homolog in disulfide bond formation in the cyanobacterium Synechocystis 6803 (30) became available.

The predictions also led to the testing of the hypothesis that some bacteria have a cell envelope in which disulfide bond formation does not occur. The results of these experiments with one of these organisms, Bacteroides fragilis, are consistent with this proposal. Further evidence is the finding that B. fragilis, as well other bacteria that are predicted to lack protein disulfide bonds, encode homologs of E. coli alkaline phosphatase that lack cysteines, in particular, those responsible for stabilizing the E. coli enzyme (31). The absence of homologs of the DsbA.DsbB system may be a satisfactory explanation for a lack of disulfide bond formation in this organism.

The presence of free cysteines in the exported proteins of organisms that do not make disulfide bonds may result in these proteins being susceptible to oxidative damage, such as sulfenic acid formation or inappropriate disulfide bond formation. This would be particularly problematic for the aerotolerant anaerobes (e.g. Bacteroides spp., Lactobacillus spp.) and the aerobic bacteria (e.g. Flavobacterium johnsoniae, Bacillus subtilis) that may lack disulfide bonds, yet survive in the presence of oxygen. Whereas cytoplasmic pathways for the repair of oxidative damage to cysteine-containing proteins have been extensively studied (32, 33), few examples of cell envelope pathways dedicated to repair of oxidized cysteines have been identified. Thus, such bacteria may have mechanisms in the cell envelope to prevent or repair oxidative damage to cysteine-containing proteins. Previous studies showed that B. fragilis has at least one pathway, the Batl pathway, that contributes to aerotolerance via the reduction of disulfide bonds in the cell envelope (34). In addition, it was found that B. fragilis and F. johnsoniae have several exported thioredoxins and thioredoxin-like proteins of unknown function that could play a role in preventing oxidative damage of cell envelope proteins. Some bacteria may have evolved other mechanisms to prevent unwanted cysteine oxidation in exported proteins. For example, it was found that the Firmicutes tend to include very little cysteine at all in exported proteins.

Since one of the major roles of disulfide bonds is believed to be the stabilization of exported proteins in the face of a fluctuating extracellular environment, it will be interesting to see if novel mechanisms of protein stabilization have evolved in bacteria that lack disulfide bond formation. Recently, the crystal structure of a pilin protein from one of the Lactobacillales, Streptococcus pyogenes, revealed a novel isopeptide bond proposed to be an alternative to disulfide bonds (35).

While not yet tested, our analysis also led to the prediction that those bacteria such as Geobacter metallireducens containing dsbA/dsbB homologs in an operon , may use the disulfide bond-forming enzymes for only a small number of specific substrates. In contrast, most of the various Proteobacteria contain dsbA and dsbB genes at different positions on their chromosomes and are predicted by this analysis to contain disulfide bonds in a high proportion of cell envelope proteins.

Lastly, these results show that in some cases the capacity for disulfide bond formation correlates with the environmental niche of the bacterium. The obligate anaerobes and obligate intracellular bacteria, which often live in reducing environments, generally are predicted by our analysis not to make disulfide bonds. This potential connection between the redox biology of cysteine-containing proteins in the bacterial cell envelope and the habitat of the bacterium may signal an interesting example of the evolution of bacterial genomes and protein folding with respect to particular environments.

### Methods

All 375 Genbank complete bacterial genomes available on Nov. 5, 2006 were downloaded. Flavobacterium johnsonii UW101 was added on May 8, 2007. The protein sequences of these genomes was analyzesd with Phobius, http://phobius.sbc.su.se/(36) and a Prosite profile for lipoproteins, release 20.0, http://www.expasy.ch/prosite/(37). Phobius is a subcellular localization prediction program based on SignalP 3.0 and TMHMM 2.0. Thus, proteins exported by the general secretion machinery, SecYEG, should be detected, as well as many proteins that are exported by the major alternative pathway for export in many bacteria, the TAT pathway(38), which utilizes signal sequences that are very similar to Sec pathway signal sequences. The Sec system is universally conserved and signals in secreted and transmembrane proteins that determine secretion and topology are similar across all bacteria. Such signals, as identified by the methods we employed, are variable but always a significant fraction of all the proteins in each bacterial genome. Thus this approach is believed adequate for estimation of gross statistical features of the distribution of cysteine residues in exported proteins of most, if not all organisms.

For each protein, each amino acid residue was assigned to one of 6 classes, based on its predicted subcellular localization. Thus, each amino acid within a protein was assigned to one of the following classes: 1. Cytoplasmic, 2. Transmembrane protein-cytoplasmic domains, 3. Transmembrane protein-inner membrane spanning helices, 4. Transmembrane protein-periplasmic domains, 5. Exported protein, directed by a signal sequence whether the final destination is the periplasm, the outer membrane or outside of the cell, and 6. Other, which includes residues predicted to be in cleavable signal sequences and the amino terminal cysteine residues of mature lipoproteins. Transmembrane proteins with predicted signal sequences were classified as transmembrane.

For each genome, two numbers for each of the twenty amino acids in each class were calculated. The first is the even fraction, the fraction of proteins with even numbers of that amino acid of that class, excluding proteins with none of that amino acid in that class. We term that number the even fraction, or Efrac. The second number, the AApref, is a measure of the preference for or bias against that amino acid in that class. This is calculated from the amino acid composition of the class and the amino acid composition of the whole proteome. It is the ratio of the frequency of the amino acid in the class to the frequency in the genome. This is the same as the ratio of the fraction of the amino acid that is in the class to the fraction of all amino acids that is in the class.

To assess the significance of the even fraction, we carried out a randomization procedure to obtain a mean value and standard deviation for the even fraction of each amino acid expected at random. The methods used for this are described in the supplemental material.

Hidden Markov models for DsbA, DsbB and VKOR were obtained from Pfam 22.0, http://pfam.sanger.ac.uk/(39). Searches were run with HMMER 2.3.2 obtained from http://hmmer.janelia.org/, and results above the significance cutoff from the ls_C file were used. DsbA homologs with a cytoplasmic localization, based on Phobius predictions, were excluded. Since the Pfam DsbB HMM model missed some known DsbB homologs found in the alpha-proteobacteria, we built an additional DsbB HMM model (based on alpha-proteobacterial DsbB sequences) to supplement the homology searches. BLASTP (40) was also used to identify additional DsbA homologs using the Staphylococcus aureus DsbA (gi11935158) as a query, and collected hits below the e-value <10-4. Information about the biology of the organisms was obtained from NCBI, http://www.ncbi.nlm.nih.gov/genomes/lproks.cgi and the Genomes Online Database, http:www.genomesoline.org/. The phylogenetic tree in FIG. 3 was generated using the Interactive Tree of Life (iTOL) web server, http://itol.embl.de (41).

The Mycobacterium tuberculosis H37Rv VKOR homolog, Rv2968c, was PCR amplified with the following primers, AGCCATGGTTGCAGCGCGACCTGCCGAGCGATCC (SEQ ID NO: 3) and CTGCAGTCTAGATCAGATCAGCGTCGACCAAT (SEQ ID NO: 4), and cloned in pDSW206 (42). Motility tests were performed on M63 minimal medium(43), with 0.3% agar, 0.2% glucose, 1mM isopropyl thiogalactoside, IPTG, and 0.2mg/m1 ampicillin, and incubated for 3 days at 30° C.

The in vivo redox state of E. coli LivK-myc when expressed in B. fragilis, was determined with B. fragilis grown anaerobically at 37C in Basal medium(44), without adding cysteine. LivK-myc was expressed from the plasmid pFD340(45). Samples were acid trapped, and then either treated with dithiothreitol (DTT) or alkylated with MalPEG-2000MW (Sunbright ME-020MA, NOF Corporation), as described previously(46). Samples were separated with SDS-PAGE, then detected using antibodies against the Myc epitope (Santa Cruz Biotechnology, Inc., Santa Cruz, Calif.).

### Additional Methods

To assess the significance of the even fraction, a randomization procedure was carried out to obtain a mean value and standard deviation for the even fraction of each amino acid expected at random. Two different randomization procedures were used for E. coli. In the first, the sequences of each class were simply randomized keeping the overall amino acid composition of the class constant. By repeating this procedure 1000 times and averaging the even fractions mean and standard deviation values were obtained. Repetition of the entire process produced identical or nearly identical results. In the second method, random numbers generated according to the Poisson distribution were used to get counts for each protein. The Poisson parameter, lambda, was set to the number of amino acids of that protein in that class times the frequency of the amino acid in the class. Again repetition for 1000 times and averaging gave a mean and standard deviation which was use to calculate a z score, the number of standard deviations between the random mean and the observed value of the even fraction. A Perl interface to the C library, RANDLIB, obtained from Comprehensive Perl Archive Network, http://www.cpan.org/was used. This method gave the same result as that described above for E. coli and was used to all other genomes since it is computationally faster.

The protein sequences of these genomes were analyzed with Phobius, http://phobius.sbc.su.se (Kali L, Krogh A, Sonnhammer E L (2007) Advantages of combined transmembrane topology and signal peptide prediction—The Phobius web server. Nucleic Acids Res 35:W429-W432) and a Prosite profile for lipoproteins, release 20.0, www.expasy.ch/prosite (Hulo N, et al. (2006) The PROSITE database. Nucleic Acids Res 34:D227-D230). Phobius is a subcellular localization prediction program based on SignalP 3.0 and TMHMM 2.0. Thus, proteins exported by the general secretion machinery, SecYEG, should be detected, as well as many proteins that are exported by the major alternative pathway for export in many bacteria, the TAT pathway (Lee P A, Tullman-Ercek D, Georgiou G (2006) The bacterial twin-arginine translocation pathway. Annu Rev Microbiol 60:373-395), which utilizes signal sequences that are very similar to Sec pathway signal sequences. The Sec system is universally conserved and signals in secreted and transmembrane proteins that determine secretion and topology are similar across all bacteria. Such signals, as identified by the methods herein, are variable but always a significant fraction of all of the proteins in each bacterial genome. Thus this approach is adequate for estimation of gross statistical features of the distribution of cysteine residues in exported proteins of most, if not all organisms.

For each protein, each amino acid residue was assigned to one of six classes, based on its predicted subcellular localization. Thus, each amino acid within a protein was assigned to one of the following classes: cytoplasmic (class 1); transmembrane protein-cytoplasmic domains (class 2); transmembrane proteininner membrane spanning helices (class 3); transmembrane protein-periplasmic domains (class 4); exported protein, directed by a signal sequence whether the final destination is the periplasm, the outer membrane or outside of the cell (class 5); and other, which includes residues predicted to be in cleavable signal sequences and the amino terminal cysteine residues of mature lipoproteins (class 6). Transmembrane proteins with predicted signal sequences were classified as transmembrane. For each genome, two numbers for each of the twenty amino acids in each class were calculated. The first is the even fraction, the fraction of proteins with even numbers of that amino acid of that class, excluding proteins with none of that amino acid in that class. That number is termed herein the even fraction, or Efrac. The second number, the AApref, is a measure of the preference for or bias against that amino acid in that class. This is calculated from the amino acid composition of the class and the amino acid composition of the whole proteome. It is the ratio of the frequency of the amino acid in the class to the frequency in the genome. This is the same as the ratio of the fraction of the amino acid that is in the class to the fraction of all amino acids that is in the class.

To assess the significance of the Efrac, a randomization procedure was carried out to obtain a mean value and standard deviation for the Efrac of each amino acid expected at random. Two different randomization procedures for E. coli were used. In the first, the sequences of each class were simply randomized, keeping the overall amino acid composition of the class constant. By repeating this procedure 1,000 times and averaging the even fractions, mean and standard deviation values were obtained.

Repetition of the entire process produced identical or nearly identical results. In the second method, random numbers generated according to the Poisson distribution were used to get counts for each protein. The Poisson parameter, lambda, was set to the number of amino acids of that protein in that class times the frequency of the amino acid in the class. Again repetition for 1000 times and averaging gave a mean and standard deviation which was use to calculate a z score, the number of standard deviations between the random mean and the observed value of the Efrac. A Perl interface to the C library, RANDLIB, obtained from Comprehensive Perl Archive Network, www.cpan.org, was used. This method gave the same result as that described above for E. coli and was used to all other genomes since it is computationally faster.

DsbA homologs with a cytoplasmic localization, based on Phobius predictions, were excluded. Since the Pfam DsbBHMM model missed some known DsbB homologs found in the lphaproteobacteria, an additional DsbBHMMmodel (based on alpha-proteobacterial DsbB sequences) was built to supplement the homology searches. BLASTP (Altschul S F, et al. (1990) Basic local alignment search tool. J Mol Biol 215:403-410) was also used to identify additional DsbA homologs using the Staphylococcus aureus DsbA (gi11935158) as a query and collected hits below the evalue of <10-4.

### REFERENCES

1. Nakamoto, H & Bardwell, J C (2004) Catalysis of disulfide bond formation and isomerization in the Escherichia coli periplasm. Biochim Biophys Acta 1694:111-9.
2. Akiyama, Y, Kamitani, S, Kusukawa, N & Ito, K (1992) In vitro catalysis of oxidative folding of disulfide-bonded proteins by the Escherichia coli dsbA (ppfA) gene product. J Biol Chem 267:22440-5.
3. Bardwell, J C, McGovern, K & Beckwith, J (1991) Identification of a protein required for disulfide bond formation in vivo. Cell 67:581-9.
4. Bardwell, J C, et al. (1993) A pathway for disulfide bond formation in vivo. Proc Natl Acad Sci USA 90:1038-42.
5. Bader, M, et al. (1999) Oxidative protein folding is driven by the electron transport system. Cell 98:217-27.
6. Kobayashi, T, et al. (1997) Respiratory chain is required to maintain oxidized states of the DsbA-DsbB disulfide bond formation system in aerobically growing Escherichia coli cells. Proc Natl Acad Sci USA 94:11857-62.
7. Mallick, P, Boutz, D R, Eisenberg, D & Yeates, T O (2002) Genomic evidence that the intracellular proteins of archaeal microbes contain disulfide bonds. Proc Natl Acad Sci USA 99:9679-84.
8. Sone, M, Kishigami, S, Yoshihisa, T & Ito, K (1997) Roles of disulfide bonds in bacterial alkaline phosphatase. J Biol Chem 272:6174-8.
9. Rietsch, A, Belin, D, Martin, N & Beckwith, J (1996) An in vivo pathway for disulfide bond isomerization in Escherichia coli. Proc Natl Acad Sci USA 93:13048-53.
10. Schwaller, M, Wilkinson, B & Gilbert, H F (2003) Reduction-reoxidation cycles contribute to catalysis of disulfide isomerization by protein-disulfide isomerase. J Biol Chem 278:7154-9.
11. Pollock, M R & Richmond, M H (1962) Low cyst(e)ine content of bacterial extracellular proteins: its possible physiological significance. Nature 194:446-9.
12. Kadokura, H, Katzen, F & Beckwith, J (2003) Protein disulfide bond formation in prokaryotes. Annu Rev Biochem 72:111-35.
13. Sevier, C S, et al. (2005) The prokaryotic enzyme DsbB may share key structural features with eukaryotic disulfide bond forming oxidoreductases. Protein Sci 14:1630-42.
14. Goulding, C W, et al. (2004) Gram-positive DsbE proteins function differently from Gram-negative DsbE homologs. A structure to function analysis of DsbE from Mycobacterium tuberculosis. J Biol Chem 279:3516-24.
15. Wiker, H G, et al. (1998) Immunochemical characterization of the MPB70/80 and MPB83 proteins of Mycobacterium bovis. Infect Immun 66:1445-52.
16. Matoba, Y, Katsube, Y & Sugiyama, M (2002) The crystal structure of prokaryotic phospholipase A2. J Biol Chem 277:20059-69.
17. Li, T, et al. (2004) Identification of the gene for vitamin K epoxide reductase. Nature 427:541-4.
18. Rost, S, et al. (2004) Mutations in VKORC1 cause warfarin resistance and multiple coagulation factor deficiency type 2. Nature 427:537-41.
19. Rost, S, et al. (2005) Site-directed mutagenesis of coumarin-type anticoagulant-sensitive VKORC1: evidence that highly conserved amino acids define structural requirements for enzymatic activity and inhibition by warfarin. Thromb Haemost 94:780-6.
20. Goodstadt, L & Ponting, C P (2004) Vitamin K epoxide reductase: homology, active site and catalytic mechanism. Trends Biochem Sci 29:289-92.
21. Wajih, N, Hutson, S M & Wallin, R (2007) Disulfide-dependent protein folding is linked to operation of the vitamin K cycle in the endoplasmic reticulum. A protein disulfide isomerase-VKORC1 redox enzyme complex appears to be responsible for vitamin K1 2,3-epoxide reduction. J Biol Chem 282:2626-35.
22. Weissman, J S & Kim, P S (1993) Efficient catalysis of disulphide bond rearrangements by protein disulphide isomerase. Nature 365:185-8.
23. Kadokura, H, et al. (2004) Snapshots of DsbA in action: detection of proteins in the process of oxidative folding. Science 303:534-7.
24. Madigan, M, Martinko, J. (2006) Brock biology of microorganisms, 11th edition (Pearson Prentice Hall, Upper Saddle River, N.J.).
25. Dorenbos, R, et al. (2002) Thiol-disulfide oxidoreductases are essential for the production of the lantibiotic sublancin 168. J Biol Chem 277:16682-8.
26. Meima, R, et al. (2002) The bdbDC operon of Bacillus subtilis encodes thiol-disulfide oxidoreductases required for competence development. J Biol Chem 277:6994-7001.
27. Yen, T Y, Pal, S & de la Maza, L M (2005) Characterization of the disulfide bonds and free cysteine residues of the Chlamydia trachomatis mouse pneumonitis major outer membrane protein. Biochemistry 44:6250-6.
28. Mac, T T, et al. (2008) Insight into disulfide bond catalysis in Chlamydia from the structure and function of DsbH, a novel oxidoreductase. J Biol Chem 283:824-32.
29. Kwon, A R & Choi, E C (2005) Role of disulfide bond of arylsulfate sulfotransferase in the catalytic activity. Arch Pharm Res 28:561-5.
30. Singh, A K, Bhattacharyya-Pakrasi, M & Pakrasi, H B (2008) Identification of an atypical membrane protein involved in the formation of protein disulfide bonds in oxygenic photosynthetic organisms. J Biol Chem.
31. Derman, A I & Beckwith, J (1991) Escherichia coli alkaline phosphatase fails to acquire disulfide bonds when retained in the cytoplasm. J Bacteriol 173:7719-22.
32. Prinz, W A, Aslund, F, Holmgren, A & Beckwith, J (1997) The role of the thioredoxin and glutaredoxin pathways in reducing protein disulfide bonds in the Escherichia coli cytoplasm. J Biol Chem 272:15661-7.
33. Poole, L B (2005) Bacterial defenses against oxidants: mechanistic features of cysteine-based peroxidases and their flavoprotein reductases. Arch Biochem Biophys 433:240-54.
34. Tang, Y P, Dallas, M M & Malamy, M H (1999) Characterization of the Batl (Bacteroides aerotolerance) operon in Bacteroides fragilis: isolation of a B. fragilis mutant with reduced aerotolerance and impaired growth in in vivo model systems. Mol Microbiol 32:139-49.
35. Kang, H J, et al. (2007) Stabilizing isopeptide bonds revealed in gram-positive bacterial pilus structure. Science 318:1625-8.
36. Kall, L, Krogh, A & Sonnhammer, E L (2007) Advantages of combined transmembrane topology and signal peptide prediction--the Phobius web server. Nucleic Acids Res 35:W429-32.
37. Hulo, N, et al. (2006) The PROSITE database. Nucleic Acids Res 34:D227-30.
38. Lee, P A, Tullman-Ercek, D & Georgiou, G (2006) The bacterial twin-arginine translocation pathway. Annu Rev Microbiol 60:373-95.
39. Finn, R D, et al. (2006) Pfam: clans, web tools and services. Nucleic Acids Res 34:D247-51.
40. Altschul, S F, et al. (1990) Basic local alignment search tool. J Mol Biol 215:403-10.
41. Letunic, I & Bork, P (2007) Interactive Tree Of Life (iTOL): an online tool for phylogenetic tree display and annotation. Bioinformatics 23:127-8.
42. Weiss, D S, et al. (1999) Localization of FtsI (PBP3) to the septal ring requires its membrane anchor, the Z ring, FtsA, FtsQ, and FtsL. J Bacteriol 181:508-20.
43. Silhavy, T J, Berman, M. L., Enquist, L. W. (1984) in Experiments with Gene Fusions (Cold Spring Harbor Laboratories, Cold Spring Harbor, N.Y.), pp. 102-112.
44. Pantosti, A, Tzianabos, A O, Onderdonk, A B & Kasper, D L (1991) Immunochemical characterization of two surface polysaccharides of Bacteroides fragilis. Infect Immun 59:2075-82.
45. Smith, C J, Rogers, M B & McKee, M L (1992) Heterologous gene expression in Bacteroides fragilis. Plasmid 27:141-54.
46. Kobayashi, T & Ito, K (1999) Respiratory chain strongly oxidizes the CXXC motif of DsbB in the Escherichia coli disulfide bond formation pathway. Embo J 18:1192-8.

### Example 2

Two significant health problems were appraoched: 1) the increasing antibiotic resistance of tuberculosis; 2) the need for constant monitoring of patients taking the anticoagulant warfarin (Coumadin). These problems were connected by the discovery that the tuberculosis bacterium, Mycobacterium tuberculosis, makes a protein, essential for growth, closely related to the target of warfarin in humans, VKOR. The tuberculosis VKOR helps proteins to fold by promoting the formation of an important chemical bond—the disulfide bond. Sensitive assay systems were developed for the identification and development of inhibitors of VKOR as it was found that tuberculosis VKOR also works in another bacterium, Escherichia coli, in which it is not essential for growth. Warfarin has been shown to inhibit the tuberculosis VKOR activity in E. coli, thereby validating our assay system. Human VKOR in E. coli will be similarly used in the E. coli assay system to help to further identify inhibitors of bacterial VKOR that do not detrimentally inhibit human VKOR. Human VKOR in E. coli will also be similarly used in the E. coli assay system to identify inhibitors of human VKOR that can be used as therapeutic anti-coagulation factors.

A large library of chemicals will be tested for inhibition of the tuberculosis VKOR in E. coli. Chemicals showing inhibition will be tested for inhibition of M. tuberculosis growth and inhibition of human VKOR activity. Inhibitors will distinguished that act on human VKOR but not tuberculosis VKOR and vice versa. The biological role and structure of Mycobacterium tuberculosis VKOR will be determined. This work will assist in developing potential VKOR inhibitors for treating tuberculosis and inhibiting blood coagulation in humans. Chemicals which inhibit vitamin K epoxide reductase (VKOR) and its homologues expressed in E. coli will allow development of new antibiotics against tuberculosis and of new classes of anticoagulants for prevention and/or treatment of thrombosis.

### Disulfide Bond Formation in the Bacterial Kingdom

A multi-faceted bioinformatic approach to mine the genome sequences of ˜400 bacterial genomes was used to determine the disulfide bond-making capacity of each bacteria and whether they use the same enzymes as E. coli. The results suggest that a majority of bacteria make disulfide-bonded proteins, some make only a few such proteins, and a significant minority do not make disulfide bonds. While a majority of bacteria contain homologues of DsbA and DsbB, a significant number contain DsbA, but not DsbB. In examining the genomes of organisms lacking DsbB, most had, directly adjacent to their potential dsbA gene, a gene that encoded a homologue of the mammalian vitamin K epoxide reductase (VKOR). Vertebrate VKOR plays a critical role in the vitamin K cycle by salvaging vitamin K epoxide and reducing it to the quinine form of vitamin K, essential for blood clotting. The role of mammalian VKOR in transferring electrons from PDI in the ER to vitamin K (a quinone) is similar to the activity of DsbB in E. coli. It was hypothesized that VKOR is the counterpart of DsbB in bacteria containing a VKOR gene.

Bacterial VKOR Performs the same Reaction as DsbB

The VKOR homologue of Mycobacterium tuberculosis (Mtb) was used to verify its role in disulfide bond formation. A genomic analysis to identify essential genes in Mtb indicated that VKOR may be essential. The Mtb VKOR gene is located adjacent to a gene for a thioredoxin-like protein. Whether VKOR could replace DsbB in the oxidation of DsbA in E. coli was investigaed. VKOR was cloned under a regulatable promoter into a dsbB-E. coli. The Mtb VKOR restored efficient disulfide bond formation to the E. coli dsbB-mutant, but does not do so to a dsbB-,dsbA-mutant. This showed that VKOR is restoring the ability of the bacteria to use DsbA for disulfide bond formation. Similar complementation with VKOR homologues from organisms as distant from E. coli as the Archaea was also observed.

Warfarin (Coumadin), the widely used oral anticoagulant, whose mode of action is directed at the inhibition of mammalian VKOR, was also shown to inhibit bacterial VKOR: The finding that sodium warfarin inhibits growth of the bVKOR expressing Mycobacterium smegmatis (Msmeg), a relative of M. tuberculosis led to the investigation of whether the growth inhibition was due to inhibition of VKOR. Since there was no known activity for VKOR in its native host, the first approach was to ask whether mycobacterial VKOR is inhibited by warfarin when it was expressed in E. coli, replacing the endogenous E. coli protein DsbB (similarly involved in disulfide bond formation in E. coli). Facilitating this study was the fact that disulfide bond formation is not essential for growth of E. coli. Therefore, the effects of warfarin on VKOR were assessed through its effects on disulfide bond formation during growth using a number of different phenotypic and protein-based assays we have developed over the years. Warfarin was found to inhibit disulfide bond formation when VKOR but not DsbB was the oxidant of DsbA.

Mutants of E. coli expressing mycobacterial VKOR that were resistant to warfarin were sought by mutagenizing the plasmid that encoded VKOR. Three such mutants were obtained. One of the mutants had an analogous mutation as a mutation found in human VKOR in certain Ashkenazi Jews, thought to cause resistance to warfarin as an oral antcoagulant. These results suggest a remarkable retaining of properties between the bacterial and human VKOR' s.

The levels of warfarin necessary to inhibit VKOR activity in E. coli and growth in Msmeg were high (millimolar concs.). This may be due to problems of entry into the bacterial cell or represent quite different drug sensitivities of the human and bacterial VKORs. The essentiality of VKOR for growth of mycobacteria, the sensitivity of mycobacterial VKOR (in E. coli) to warfarin, and the ability to sensitively measure effects of inhibitors of mycobacterial VKOR in E. coli provide us with productive tools for seeking 1) new classes of antibiotics towards Mtb and 2) new classes of inhibitors of VKOR that might have advantages over warfarin.

Establishing that VKOR is Essential in Mycobacteria

The method used to previously indicate that VKOR is essential in M. tuberculosis was an indirect one. This indirect evidence that bacterial VKOR is essential comes from a study which used a high-throughput method to mutagenize M. tuberculosis with transposons (Sassetti et al., Mol Microbiol. 2003 April; 48(1):77-84). These transposons should interrupt most genes on the chromosome, with one gene interrupted per bacterium. After the mutagenesis, the bacteria were allowed to grow, and the location of the transposon in the genome of each growing bacterium was identified. If a transposon was not found in any given gene, it suggested that the bacteria that had that mutation were not able to grow—thus, the gene was essential for growth. Rv2968c, the gene encoding the M. tuberculosis VKOR, was found to lack transposon by this method. So was Rv2969c, a gene predicted to be in the same operon as VKOR, and which was hypothesized to act in the same biological pathway as VKOR. However, this method is an indirect method of identifying candidate essential genes. It is possible that no transposons were found in Rv2968c because it is small, not because it is essential.

More direct evidence establish that VKOR will serve as an effective target for development of an antimicrobial agent will be obtained using well-established techniques in mycobacteria for generating deletions of genes. The VKOR deletion will be constructed in Msmeg in the presence of a plasmid expressing VKOR regulated from a tetracycline-inducible promoter. Essentiality of the VKOR gene will be directly indicated by the deletion strain's growth depending on expression of the plasmid-encoded VKOR. A second approach is to express the warfarin-resistant mutants of Mtb VKOR (obtained in E. coli) in Msmeg to restore growth to the bacteria in the presence of growth-inhibitory concentrations of warfarin.

To establish that VKOR is required for disulfide bond formation in Msmeg, the formation of disulfide bonds in the mycobacterial alkaline phosphatase will be assessed either in cells as they are depleted of VKOR in the strains obtained in the preceding paragraph or after the inhibition of VKOR by warfarin. In addition, genes that are candidates for the Msmeg DsbA counterpart will be cloned and will be tested on their ability to oxidize E. coli substrate proteins with VKOR present. Such candidate gene(s) will be deleted in Msmeg to assess their effects on disulfide bond formation using depletion strains if the DsbA-like gene is essential.

A plasmid to make a deletion of the VKOR homolog in the related bacterium Mycobacterium smegmatis, will be constructed in order to provide further support for the essentiality of VKOR to these bacteria. Deletion of VKOR should will prohibit growth, or at least severely impair the growth of M. smegmatis, indicating that VKOR is a good target for an antimicrobial agent.

### Obtaining Inhibitors of Mtb VKOR

Work over the years with DsbA has provided a very sensitive assay for inhibitors of disulfide bond formation. A fusion of E. coli β-galactosidase (β-gal) to a cytoplasmic membrane protein (MalF) causes the β-gal to be translocated into the bacterial periplasm. There, DsbA joins β-gal cysteines in disulfide bonds resulting in an inactive enzyme. Slight defects in DsbA or DsbB alleviate the disulfide bond formation and allow a fraction of the β-gal enzyme molecules to be active. The β-gal activities can vary over a 1500-fold range from fully active (in a DsbA− or B−) to inactive (in a DsbA+ or B+). Thus, in an E. coli strain carrying the β-gal fusion and which has Mtb VKOR instead of DsbB, inhibitors of VKOR can be screened for by seeking chemicals that cause an increase in β-gal activity. Using this MalF-β-gal fusion, effects of a wide variety of chemicals on levels of β-gal activity will be compared in a wild-type (DsbB+) strain, a strain containing Mtb VKOR instead of DsbB. Effects of such chemicals on levels of β-gal activity will also be compared in a wild-type (DsbB+) strain containing human VKOR instead of DsbB. This assay will be performed in multi-well plates and inhibition compared to a strain that carries DsbB instead of VKOR. The enormous advantage of this screen compared to many screens for chemical inhibitors is that we are not screening for killing of the bacteria, which could be due to a host of causes, but instead are screening for inhibition of a specific protein's activity which happens to be essential for growth in Mtb but not in E. coli. The Institute for Chemistry and Chemical Biology (ICCB) at Harvard Medical School will carry out such a screen with portions of their collection of 250,000 chemicals. Chemicals that are identified to inhibit Mtb VKOR will then be tested to identify chemicals that inhibit the growth of Mtb bacteria themselves.

Assessing Human VKOR Activity in E. coli

Proteins that use thiol-redox activities in reductive or oxidative processes are often interchangeable between widely different organisms. This is shown by 1) the finding that VKORs from a range of micro-organisms can replace DsbB and 2) studies which show that eukaryotic enzymes such as PDI, thioredoxins or glutaredoxins are functional in E. coli. On this basis, human VKOR, which carries out analogous reactions to DsbB, will be able to substitute for DsbB in E. coli. A synthesized gene encoding human VKOR that is codon-optimized for expression in E. coli, which will be tested for restoration of disulfide bond formation.

Human VKOR is expected to restore disulfide bond formation to a dsbB mutant. Human VKOR in this system will be used to distinguish inhibitors of Mtb that also inhibit human VKOR. Such inhibitors are less desireable because they could cause anticoagulation in patients while being used to treat tuberculosis or other bacterialVKOR expressing pathogens.

The sensitive assay of inhibitors of bacterial VKOR (motility, β-gal, etc.) will be used in parallel assays with E. coli strains expressing Mtb VKOR and human VKOR. At least two useful classes of inhibitors will be obtained from such a screen. 1) inhibitors that inhibit MtbVKOR but do not (or at least have a substantially lower inhibitory effect on) human VKOR. These are potential antibiotics for tuberculosis treatment, as well as treatment of other VKOR expressing pathogens. And 2) inhibitors that act on human VKOR as potential new anti-coagulant drugs. The effects on vitamin K epoxide reductase activity of potential inhibitors obtained with the ICCB tests will be assessed to identify each class of inhibitor. In vitro assays will be performed that will monitor vitamin K generation from vitamin K epoxide via HPLC. Particularly promising inhibitors will be modified chemically to seek more effective inhibitors of either human or Mtb VKOR or both.

### Bacterial VKOR is Sensitive to Warfarin

The above discussed assays have been used to identify warfarin as an inhibitor of bVKOR. After VKOR was identified using bioinformatics as a possible disulfide bond formation protein in M. tuberculosis (and many related bacteria), this protein was expressed in E. coli in order to test if it could catalyze the formation of disulfide bonds. A number of assays have been developed in E. coli that are sensitive to disulfide bond formation.

Assays have been developed in E. coli to measure the activity of bacterial VKOR, as well as inhibition of VKOR activity. Motility has been primarily measured and the activity of β-galactosidase fused to the membrane protein MalF (malF-lacZ fusion). However, similar results with other methods, including alkaline phosphatase assays and direct alkylation of proteins to examine their redox state (i.e. determine if disulfide bonds are formed) have been observed.

In all experiments, a strain of E. coli in which the gene encoding DsbB has been deleted is used. This strain is unable to make disulfide bonds, because DsbB is normally required for this process. Then, a plasmid that expresses bacterial VKOR (in this case, MtbVKOR) is placed into this strain. When VKOR is expressed, it replaces the function of DsbB, and the bacteria can now make disulfide bonds—as measured by the assays.

In the motility assay, this was observed as an increase in motility. Since it was hypothesized that bacterial VKOR would have a similar function to the E. coli protein DsbB, the M. tuberculosis VKOR (MtbVKOR) was expressed in E. coli that was missing DsbB and then assayed for production of disulfide bonds. In this case, a motility of the bacteria as the assay was used, since motility is dependent on disulfide bond formation. MtbVKOR complemented the E. coli missing DsbB, and thus that it could catalyze disulfide bond formation (Dutton et al., Proc Natl Acad Sci USA. 2008 Aug. 19; 105(33):11933-8.).

For the β-galactosidase assay, the malF-lacZ fusion has been placed on the chromosome in the E. coli strains. In this assay, an absence of disulfide bond formation (as in the DsbB− strain) results in high activity of the β-galactosidase. When VKOR is expressed in this strain, disulfide bond formation is restored, and a low activity of β-galctosidase was observed. When warfarin was added to the strain expressing VKOR, the activity of the β-galactosidase significantly increased, indicating an inhibition of disulfide bond formation. It is important to note that although VKOR appears to be essential in mycobacteria, inhibition of VKOR expressed in E. coli does not inhibit the growth of E. coli.

It is proposed that the β-galactosidase activity from the malF-lacZ fusion strains will be particularly useful in screening for novel inhibitors of bacterial and/or human VKOR. This is because inhibition of VKOR in this assay leads to an increase in β-galactosidase activity. Thus, this is a positive read-out for inhibition, or a gain of function upon inhibition. This will eliminate many false positives from compounds that kill or inhibit growth of the bacterium, instead of specifically targeting VKOR.

Since human VKOR is known to be inhibited by the anti-coagulant drug warfarin (also known as Coumadin), it was asked whether the bacterial VKOR was also sensitive to the drug. Using the motility assay, as well as several other assays, whether addition of warfarin to wild type E. coli and E. coli expressing MtbVKOR resulted in a decrease in disulfide bond formation was investigated. Addition of warfarin to the wild type E. coli had no effect in the assays; however warfarin decreased the ability of E. coli expressing MtbVKOR to make disulfide bonds. These experiments show that warfarin does indeed inhibit bacterial VKOR.

Since VKOR was found to be an essential gene in M. tuberculosis (in the study mentioned above, Sassetti et al.), it was reasoned that inhibition of the VKOR protein by addition of warfarin may be lethal for mycobacteria. Indeed, addition of warfarin to a culture of M. smegmatis caused inhibition of growth. The minimal inhibitory concentration of warfarin toward M. smegmatis was determined using an alamar blue assay, and was found to be 4.5 mM when the bacterium were grown in Middlebrook 7H9 medium (defined medium) and 2.25 mM when grown in NZ-glucose (rich medium).

While some data indicates that bacterial VKOR is inhibited by warfarin (based on results in E. coli), it is possible that warfarin has other targets in mycobacteria that result in growth inhibition. Warfarin-resistant mutants of MtbVKOR, have now been isolated in E. coli, which are planned to be expressed in M. smegmatis. If expression of these mutants alleviates the effects of warfarin on M. smegmatis, this would provide additional evidence that the primary target of warfarin in mycobacteria is the VKOR protein.

There have been some previous reports that compounds related to warfarin, specifically ferulenol isolated from the Sardinian Giant Fennel, inhibit the growth of some species of mycobacteria. These papers did not test the effect of warfarin on mycobacteria, and at the time it was not known that mycobacteria had a homolog the human VKOR protein. Thus, it is not known whether ferulenol and its derivatives target bacterial VKOR. However, it is known that ferulenol has anti-coagulant properties in animals (Appendino et al., Antimycobacterial coumarins from the sardinian giant fennel (Ferula communis). J Nat Prod. 2004 December; 67(12):2108-10.; Monti et al., Characterization of anti-coagulant properties of prenylated coumarin ferulenol. Biochimica et Biophysica Acta, Volume 1770, Issue 10, Pages 1437-1440).

### Human VKOR in the Assays to Screen for Inhibitory Agents

A codon-optimized version of the human VKOR obtained from Genscript for expression in E. coli will be used. This version will be verified as capable of catalyzing disulfide bond formation in E. coli useing the same types of assays used to characterize bacterial VKOR (as described above).

### Materials and Methods

### Strain and Plasmid Construction:

Cloning of bacterial VKOR: A DNA fragment containing the gene for Mycobacterium tuberculosis H37Rv VKOR was amplified from chromosomal DNA using the primers, AGCCATGGTTGCAGCGCGACCTGCCGAGCGATCC (SEQ ID NO: 3) and CTGCAGTCTAGATCAGATCAGCGTCGAACCAAT (SEQ ID NO: 5) by PCR. The PCR product was digested with NcoI and XbaI and ligated into pDSW206 (Weiss et al 1999), which had been digested with the same enzymes. The ligation product was transformed into HK325 (MC1000 ara-del714 leu+ ΔdsbB) using standard heat shock transformation methods. Subsequently the gene was subcloned into pTrc99a (Pharmacia, Piscataway, N.J.) and an oligo-histidine tag was added to the amino terminus (VKOR was cloned into pET14b in order to tag the amino terminus of the protein with a histidine tag (Novagen), and then the fragment containing the his-tagged VKOR was subcloned from pET14b using the sites NcoI and HindIII back into pTrc99a). These VKOR-containing plasmids were able to complement the dsbB− strain by restoring disulfide bond formation to E. coli.

Construction of the Strain to be used in Beta-Galactosidase Assays:

A strain in which the plasmid carrying the VKOR gene is integrated into the chromosome of E. coli was constructed, in order to ensure stable expression. Integration of the plasmid was done using the Lambda InCh1 protocol. Lambda InCh1 and methods for working with it are described in Boyd et al 2000. The E. coli strain also was deleted for dsbB, and carries the malF-lacZ fusion, allowing assays of beta-galactosidase activity. DHB7657 is the resulting strain:
DHB7657: Lambda InCh1 lad Ptrc His-MtVKOR bla/DHB7640 recA::cat. (DHB7640 HK325 Pmal-malF-lacZ:kan stabilized at the lambda attachment site. HK325 MC1000 ara-del714 leu+ dsbB-del.)

The MalF-beta-gal fusion was constructed as per Froshauer et al., J Mol Biol. 200: 501-11 (1988).

### Beta-Galactosidase Assays:

The standard assay for beta-galactosidase activity is derived from Miller 1992. The OD 600 of a log phase culture of DHB7657 growing in M63 maltose medium at 30C with 2mM IPTG was read. IPTG induces the expression of VKOR. One culture was not induced with IPTG. A second culture contained 2mM IPTG. A third culture contained 2 mM IPTG and 10 mM warfarin. A pair of 1 ml aliquots from each culture were centrifuged and resuspended in 1 ml Z-buffer. One sample of each pair was treated with chloroform (2 drops) and SDS (one drop of 0.1%) and incubated at 37C for 20 minutes. Reactions were initiated by adding 0.2 ml of 4 mg/ml ONPG and terminated by adding 0.5 ml 1M sodium carbonate. OD420 and OD550 were read on the supernatant after pelleting the cells. Units of beta-galactosidase activity were calculated by the following formula:

1000×(OD420−(1.75×OD550)/(OD600×ml of culture×minutes of assay)

Assays with and without chloroform and SDS had similar activities so the results were averaged.

The approach that will be tried first will be simply adding ONPG to the culture medium and incubating at 30C on the dark until nearly maximal color develops in the un-induced controls. Time of incubation will depend on the growth rate under the conditions of limited aeration obtained in microtiter plates. OD420 and OD550 will be read directly on the culture. Preliminary experiments with aerated 5 ml cultures indicate that usable results can be obtained after 20 to 24 hours and suggest that a somewhat shorter time of incubation might be optimal under those conditions.

| M63 medium | per liter | |
|---|---|---|
| K2HPO4 | 7 | g |
| KK2PO4 | 3 | g |
| (NH4)2SO4 | 2 | g |
| MgSO4•7H2O | 0.2 | g |
| FeSO4 | 0.5 | mg |
| maltose | 2 | g |
| thiamin | 1 | mg |

| Z-buffer | per liter | |
|---|---|---|
| NaH2PO4•7H2O | 16.1 | g |
| Na2HPO4•H2O | 5.5 | g |
| KCl | 0.75 | g |
| MgSO4•7H2O | 0.246 | g |
| 2-mercaptoethanol | 2.7 | ml |

Miller J H 1992. A Short Course in Bacterial Genetics. Cold Spring Harbor Press Cold Spring Harbor N.Y.
Weiss D, Chen J C, Ghigo J-M, Boyd D, Beckwith J. 1999 Localization of FtsI (PBP3) to the Septal Ring Requires Its Membrane Anchor, the Z Ring, FtsA, FtsQ, and FtsL. Journal of Bacteriology, 181:508-20.
Boyd D, Weiss D S, Chen J C, Beckwith J. 2000 Towards single-copy gene expression systems making gene cloning physiologically relevant: lambda InCh, a simple Escherichia coli plasmid-chromosome shuttle system. J Bacteriol. 182:842-7.

What is claimed:

1. A method for inhibiting the growth of a microbe that expresses bacterial vitamin K epoxide reductase (bVKOR), comprising contacting the bacterial cell with an effective amount of an agent that inhibits bVKOR.

2. The method of claim 1, wherein the agent does not detrimentally inhibit human (h)VKOR.

3. The method of claim 1, wherein the agent is a drug, ligand or portion thereof, protein, polypeptide, small organic molecule, antisense nucleic acid, RNAi, or antibody.

4. The method of claim 1, wherein the agent is a phenylpropanoid, a modified phenylpropanoid, a coumarin or modified coumarin.

5. The method of claim 1, wherein the agent is warfarin or a variant thereof or ferulenol or a variant thereof.

6. (canceled)

7. The method of claim 1, wherein the microbe is Mycobacterium tuberculosis.

8. (canceled)

9. A method for identifying a bVKOR inhibitory agent, comprising the steps,
a) testing one or more test agents in a disulfide bond formation assay, wherein bVKOR functions as the oxidant of DsbA in the assay; and
b) identifying test agents that significantly inhibit disulfide bond formation in the assay;
wherein the ability of the candidate agent to significantly inhibit disulfide bond formation in the assay indicates that it is a bVKOR inhibitory agent.

10. (canceled)

11. The method of claim 9, wherein the bVKOR is from M. tuberculosis.

12. The method of claim 9, further comprising testing the test agent identified in step b) in an assay for disulfide bond formation, wherein hVKOR functions as the oxidant of DsbA in the assay, to thereby identify a bVKOR inhibitory agent that does not significantly inhibit hVKOR.

13. The method of claim 9, further comprising testing the test agent identified in step b) in an assay for bVKOR activity to further indicate the test agent is a bVKOR inhibitory agent.

14. The method of claim 13, wherein the assay for bVKOR activity is a growth inhibitory assay for a microbe that naturally expresses bVKOR.

15. A method for identifying an antimicrobial agent, comprising the steps:
a) assaying one or more test agents for bVKOR inhibitory activity, and for hVKOR inhibitory activity, to thereby identify a test agent that inhibits bVKOR significantly more than it inhibits hVKOR.; and
b) further assaying the test agent identified in step a) for growth inhibition activity on a bVKOR expressing microbe, wherein an agent that exhibits growth inhibition activity on the microbe, is thereby identified as an antimicrobial agent.

16. The method of claim 15, wherein bVKOR inhibitory activity is assayed in a disulfide bond formation assay, wherein bVKOR functions as the oxidant of DsbA in the assay, and wherein hVKOR inhibitory activity is assayed in a disulfide bond formation assay, wherein hVKOR functions as the oxidant of DsbA in the assay.

17. The method of claim 15, further comprising assaying the identified test agent of step a) for anti-coagulant activity, wherein a test agent which lacks anti-coagulant activity is further assayed in step b).

18. (canceled)

19. (canceled)

20. (canceled)

21. The method of claim 15 claims 9 wherein the one or more test agents assayed is a drug, ligand or portion thereof, protein, polypeptide, small organic molecule, antisense nucleic acid, RNAi, or antibody.

22. The method of claim 15, wherein the one or more test agents assayed is a phenylpropanoid, a modified phenylpropanoid, a coumarin or modified coumarin.

23. The method of claim 15, wherein the one or more test agents assayed is warfarin or a variant thereof or ferulenol or a variant thereof.

24. The method of claim 9, wherein the disulfide bond formation assay is selected from the group consisting of a motility assay, a β-gal assay using β-gal fused to a bacterial membrane protein, and an alkaline phosphatase assay.

25. (canceled)

26. The method of claim 24, wherein the β-gal is fused to bacterial membrane protein MalF, to thereby produce a MalF-β-gal fusion protein.

27. (canceled)

28. The method of claim 16, wherein the disulfide bond formation assay is performed in E. coli.

29. (canceled)

30. (canceled)


SEQUENCE LISTING (5 sequences)

SEQ ID NO: 1 - protein, 210 aa, Mycobacterium tuberculosis
MVAARPAERSGDPAAVRVPVPSAWWVLIGGVIGLFASMTLTVEKVRILLDPIYVPSCNVNPIVSCGSVMTTPQASLLGFPNPLLGIAGFTVVVVTGVLAVAKVPLPRWYWIGLAVGILVGVAFVHWLIFQSLYRIGALCPYCMVVWAVIATLLVVVASIVFGPMRENRGSQERVGARLLYQWRWSLATLWFTTVFLLIMVRFWDYWSTLI

SEQ ID NO: 2 - DNA, 633 bp, Mycobacterium tuberculosis
gtggtggcagctcgacctgccgagcgatccggcgacccggctgcggtgcgggtgccggtgcctagcgcgtggtgggtgctgatcggcggtgtgatcgggctgttcgcttcgatgacgctcacggtggagaaggtcaggattctgctcgatccgatctatgtgccgtcatgcaatgtcaacccgatagtgtcgtgcggctcggtgatgaccacaccgcaggcgtcgttgctgggctttcccaacccgcttctcggcattgcgggcttcaccgtggtggtggtcaccggggtgctggcggtggcgaaagtgccgttgcccaggtggtattggatcggcctggcggtcgggatcctggttggtgtggcgttcgtgcattggctgatattccagagcctgtaccgtatcggtgccctgtgcccatactgcatggtggtctgggcggtcatcgccacactgctggtggtggtcgcgtccatcgttttcggtccgatgcgggaaaaccggggaagccaggagcgcgtcggggcacggctgctctatcaatggcgatggtcgctggccacgctctggttcacgacagtattcctgctgatcatggtccgcttttgggactattggtcgacgctgatctga

SEQ ID NO: 3 - DNA, 34 bp, Artificial Sequence
  Description of Artificial Sequence Synthetic primer
agccatggttgcagcgcgacctgccgagcgatcc

SEQ ID NO: 4 - DNA, 32 bp, Artificial Sequence
  Description of Artificial Sequence Synthetic primer
ctgcagtctagatcagatcagcgtcgaccaat

SEQ ID NO: 5 - DNA, 33 bp, Artificial Sequence
  Description of Artificial Sequence Synthetic primer
ctgcagtctagatcagatcagcgtcgaaccaat